(19) United States
(12) Patent Application Publication
(10) Pub. No.: US 2018/0355242 A1
(43) Pub. Date: Dec. 13, 2018

(54) OXYGEN SENSING DIFLUOROBORON B-DIKETONATE POLYLACTIDE MATERIALS FOR WOUND IMAGING

(71) Applicant: UNIVERSITY OF VIRGINIA PATENT FOUNDATION, Charlottesville, VA (US)

(72) Inventors: Cassandra L. FRASER, Charlottesville, VA (US); James N. DEMAS, Charlottesville, VA (US); Shayn PEIRCE-COTTLER, Charlottesville, VA (US); Alexander MATTHEW, Charlottesville, VA (US); Christopher DEROSA, Charlottesville, VA (US); Scott SEAMAN, Charlottesville, VA (US)

(21) Appl. No.: 16/060,761

(22) PCT Filed: Dec. 12, 2016
(86) PCT No.: PCT/US2016/066155

§ 371 Date: Jun. 8, 2018

Related U.S. Application Data
(60) Provisional application No. 62/266,162, filed on Dec. 11, 2015.
(60) Provisional application No. 62/418,499, filed on Nov. 7, 2016.

(51) Int. Cl. C09K 11/06; G01N 33/58

(52) U.S. Cl. CPC C09K 11/06; G01N 21/64; G01N 33/582

(57) ABSTRACT

Disclosed herein are methods and related imaging systems to measure oxygenation levels on a surface. Methods of monitoring wound healing with dual emissive difluoroboron naphthyl-phenyl β-diketonate polylactide materials are disclosed.

### PRIORITY

This application claims priority from U.S. Provisional Application No. 62/266,162 filed on Dec. 11, 2015, and U.S. Provisional Application No. 62/418,499 filed on Nov. 7, 2016, the disclosures of which are incorporated by reference.

### STATEMENT REGARDING FEDERALLY SPONSORED RESEARCH OR DEVELOPMENT

This invention was funded at least in part by funds from the U.S. Government (NIH Grant Nos. R01 CA167250; T32 GM008715 and P30 CA44579). The U.S. Government has certain rights in this invention.

### BACKGROUND

Oxygenation is powerful indicator of health and healing.^{1,2} Deficits and excesses in oxygen levels are associated with medical conditions such as cancer, cardiac ischemia, and chronic wounds, however oxygen levels are rarely used for diagnosis in the clinical setting. For example, although it is well known that the oxygen level within wounds is directly related to healing potential, clinical monitoring of oxygen levels is not commonly practiced largely because the current tools are invasive, expensive, rely on indirect quantification of oxygenation, and do not provide oxygenation levels within the entire wound bed. Instead, clinical assessment of wound healing is largely qualitative and can be highly subjective.² Therefore, new and effective methods for imaging and diagnosis would be valuable.³

Boron dyes are a class of responsive luminescent materials,¹³⁻¹⁷ that includes mechanofluorochromic powders,¹⁸⁻²¹ photo-switchable devices,²²⁻²⁴ ion sensors,^{25,26} and dual emissive oxygen reporters.²⁷⁻²⁹ Dual emissive difluoroboron β-diketonate poly(lactic acid) (“BF₂bdkPLA”) materials are alternative main group oxygen sensing systems that combine oxygen sensitive phosphorescence, a fluorescence standard, and a material support all in one.^{27,30}

International Patent Application Publication No. WO2011/011646 discloses difluoroboron β-diketonate (BF₂bdk) materials having both fluorescent and phosphorescent properties. The disclosed compounds and compositions in Application Publication No. WO2011/011646 are suitable for imaging and quantifying hypoxia and anoxia in cell, tissue and in vivo contexts. The BF₂bdk materials can be used for oxygen sensing or imaging of tumors, vasculature, wounds, brain imaging, high altitude drug testing, monitor drugs that delivery oxygen to tissues, organ transplantation or tissue transplantation, or cell transplantation, tissue engineering, cells, e.g., stem cells, or other tissues. BF₂bdk materials can serve as “turn on” sensors that light up in hypoxic environments such as ischemia, damaged or blocked vasculature, or are used in, e.g., fluid or gas flow and aerodynamics applications. The diagnostic imaging can provide an oxygen concentration map of tissues examined. The BF₂bdk materials can be readily processed into powders, films, particles (including nanoparticles), fibers (including nanofibers), coatings, bulk materials, gels, networks, assemblies, suspensions, composites and the like.

Boron dye-PLA biomaterials have been shown to be fabricated in myriad forms, as nanoparticles,^{31,32} nanofibers,³³ and films.²⁷ The value of these bright, photostable, nontoxic materials for biomedicine has been demonstrated. When applied to HeLa and CHO cells, boron nanoparticles (BNPs) were taken up and accumulated in perinuclear regions, as verified by fluorescence and 2-photon confocal microscopies.³² Intratumoral administration of boron dye-PLA nanoparticles in conjunction with hyperspectral imaging enabled tumor hypoxia imaging in a murine dorsal window chamber model.²⁷ When injected intravenously, PLA nanoparticles illuminated vasculature, whereas, PEG-PLA systems accumulate in tumor tissue.^{34,35} Electrospun nanofibers of boron biomaterials have found application in tissue engineering, specifically in tracking ischemia in cell transplant models.³³

Designing BF₂bdkPLA materials that combine phosphor, standard and matrix in one poses unique challenges for sensing optimization.³⁶⁻³⁹ These include: 1) enhancement of phosphorescence intensity,^{27,36-38} 2) spectral separation of fluorescence (F) and phosphorescence (P) peaks and F/P intensity tuning,^{36,37} 3) alignment of emission peaks with detection systems (e.g. common filter cubes, RGB camera channels),³⁶ and 4) photostability.³⁸ These issues are addressed by modulation of ligands, molecular weight (i.e dye loading), heavy atoms, and dye aggregation.

While a synthetic process is required to generate the O₂ sensitive dye-polymers, these main group boron dyes are less expensive and much easier to make than heavy atom Pt and Pd porphyrin dyes. Other benefits of these “all in one” materials are facile nanoparticle fabrication, and very little nanoparticle suspension is required for imaging. Though “cocktail” oxygen sensing foils can sometimes utilize commercially available dyes and polymers, they have involved fabrication to avoid problems with energy transfer and dye leaching. The covalent linkage of a dual emissive dye (BF₂bdk) to a biocompatible matrix such as PLA eliminates problems with dye leaching and energy transfer seen in the conventional two dye, sensor/standard approach.

Bromine substituted and un-substituted naphthyl-phenyl β-diketonate polylactide materials (BF₂nbm(X)PLA, where X═Br or H) have been characterized previously, and proposed as potentially useful for lifetime based imaging.^{49,88} The Br substituted napthhyl-benzyl compounds have been proposed as useful for simultaneous lifetime and ratiometric oxygen sensing and imaging.³⁶

The combination of luminescent nanomaterials and camera imaging has been used for spatiotemporal wound mapping.⁴⁻⁷ Phosphorescence imaging agents, with emission susceptible to oxygen quenching, have been used to quantify oxygen via lifetime or intensity. Typically, nanoprobes are incorporated into a polymer matrix to monitor local wound oxygenation.^{8,9} Wolfbeis and coworkers prepared a multifunctional foil comprised of oxygen sensing Pt porphyrin polystyrene microparticles, pH sensitive FITC polyacrylonitrile microparticles, with a diphenylanthracene standard on a poly(vinylidene chloride) support.⁸ With a conventional digital camera, wound pH and 0₂ levels were monitored in a clinical setting.⁹ Evans and coworkers have developed Pt and Pd porphyrin “clickaphors” with bright and tunable red phosphorescence conjugated to biocompatible poly(l-lysine) dendrimer scaffolds.^{10,11} These materials were applied as rapid drying bandages¹² or as probes for NIR oxygen mapping. With a high-speed color camera, lifetime or coumarin referenced red/green/blue (RGB) imaging, can be used to monitor wound oxygen levels. These approaches rely on barriers (e.g. Saran,⁹ Tegaderm¹²) to block or slow ambient O₂ diffusion into the sensor and tissue, and a delay time before imaging.

Covering can cause healthy tissue to become hypoxic overtime through consumption of available oxygen, while damaged tissue remains at the same oxygen level, or slightly lower based on the degree of damage. Other sensing systems typically reported percent O₂ consumed, rather than the percent oxygen present and available for consumption.

Where past methods used oxygen consumption to distinguish normal and damaged tissue, a cover-free method would enable imaging of the native oxygen environment in the wound bed and surrounding skin, with minimal perturbation and a relatively rapid readout.

It is desirable to have materials with a linear oxygen sensitivity over the full range of oxygen concentration (0 to 100%) up to ambient atmospheric conditions and beyond. For example, materials with linear oxygen sensitivity at ambient atmospheric conditions and beyond would be useful for non-invasive wound diagnosis in order to distinguish wound and keratinized skin oxygenation, even without covering the tissue.

### SUMMARY OF THE INVENTION

This invention generally relates to materials and methods for oxygen sensing. In particular, this invention provides novel iodo napththyl-phenyl difluoroboron β-diketonate compounds of Formula 1:

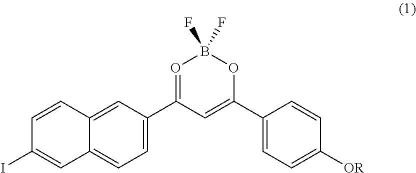

where R is selected from the group consisting of H, (C₁-C₁₂)alkyl, (C₃-C₁₂)cycloalkyl, (C₁-C₁₀)alkoxy, (C₂-C₁₂)alkenyl, (C₂-C₁₂)alkynyl, (C₁-C₁₂)alkanoyl, (C₁-C₁₂)haloalkyl, (C₁-C₁₂)hydroxyalkyl, (C₁-C₁₂)alkoxycarbonyl, (C₁-C₁₂)alkylthio, (C₂-C₁₂)alkanoyloxy, (C₆-C₂₂)aryl, (C₆-C₁₃)heteroaryl, a polymeric group or combinations thereof, and their use in monitoring oxygenation levels on surfaces. The invention also provides for compositions with compounds of Formula 1. Compounds of Formula 1 exhibit an unprecedented linear oxygen sensitivity over the full-range (0 to 100%).

Methods according to the invention provide for determining oxygenation levels on a surface by contacting the surface with compounds or compositions containing compounds of Formula 1 under ambient atmospheric conditions; exposing the compound on the surface to an excitation source under ambient atmospheric conditions; detecting the fluorescence and phosphorescence of the compound on the surface under ambient atmospheric conditions; and determining oxygenation levels on the surface based on the ratio of fluorescence to phosphorescence of the compound.

Methods according to the invention also provide for monitoring wound healing over one or more days, by determining oxygenation levels on an uncovered wound, by contacting the uncovered wound with compounds or compositions containing compounds of Formula 1; exposing the compound on the uncovered wound to an excitation source; detecting the fluorescence and phosphorescence of the compound on the uncovered wound; and determining oxygenation levels of the wound based on the ratio of fluorescence to phosphorescence of the compound.

### BRIEF DESCRIPTION OF THE DRAWINGS

FIG. 1 shows Optical properties of polymer films. Photographs of dye-polymers under air, N₂, and under N₂ with the lamp turned off (delay) and corresponding total emission spectra in air and N₂ (λₑₓ=385 nm).

FIG. 2 shows lifetime imaging with H-NPs. (A) Photographs of H-NP nanoparticles in air (F) (UV lamp on) and under N₂ (P) (UV lamp turned off). (B) Selected video frames of oxygen quenching from 0% (N₂; start) to 1% oxygen, as a steady stream of 1% O₂ (Praxair) is blown into the nanoparticle suspension.

FIG. 3 shows dual-mode Br-NP oxygen sensing. (A) Images of Br-NP in N₂ and air (B) Selected video frames of oxygen quenching from 0% (N₂) to 21% oxygen (Praxair) as a steady stream of 21% O₂ (Praxair) is blown into the nanoparticle suspension.

FIG. 4 shows ratiometric oxygen calibration with Br-NP. Stern-Volmer (F/P) plot displayed as referenced intensity (RI; fluorescence and phosphorescence peaks from the total emission spectra) (n=3).

FIG. 5 shows lifetime Stern-Volmer plot of Br-NP (n=3).

FIG. 6A shows I-NP oxygen calibration. Images of an aqueous nanoparticle suspension under oxygen, air and nitrogen (left). Total emission spectra at 0-100% O₂ (λₑₓ=385 nm; black line=0% O₂) (right). FIG. 6B shows ratiometric oxygen calibration (0-21% O₂). Stern-Volmer (F/P) plot displayed as blue channel/green channel (B/G), blue channel/red channel (B/R), referenced intensity (RI; fluorescence and phosphorescence peaks from the total emission spectra). FIG. 6C shows Stern-Volmer F/P ratiometric calibration of BF₂nbm(I)PLA nanoparticles from 0-100% oxygen (n=3).

FIG. 7 shows in vivo camera imaging with I-NPs. (A) Illustration of mouse dorsal wounds (gray circles=control wounds, blue circles=NP treated wounds; details in Figures S20 and S21). (B) Bright field image (C) Raw image under UV excitation (D) Blue channel (fluorescence). (E) Red channel (phosphorescence). (F) RGB image (blue/red channel).

FIG. 8A shows I-NP wound monitoring with daily nanoparticle application. FIG. 8B shows I-NP wound monitoring with single nanoparticle application.

FIG. 9 shows wound oxygenation and healing time course for three wounds with daily application with I-NP. Row (A) Brightfield image of wound, Row (B) uncovered ratiometric image (image taken 5 min after NP application) and Row (C) covered ratiometric image (image taken 5 min after applying glass cover slip).

FIG. 10 shows a preliminary wound study with a first-generation oxygen sensor. Nanoparticles of BF₂dbm(I)PLA (13 kDa) were used. Daily images acquired: top row=LED excitation; bottom row=green/blue ratiometric images processed via MATLAB.

FIG. 11 shows wound oxygenation and healing time course with Br-NP. Row (A) Brightfield image of wound, Row (B) uncovered ratiometric image (image taken 5 min after NP application) and Row (C) covered ratiometric image (image taken 5 min after glass cover slip).

FIG. 12 A shows the fraction of the wound bed remaining after days of healing by normalizing wound area by the original wound area. FIG. 12B shows the area under the curve for each treatment to determine the effect of NPs on wound healing. One-way ANOVA and P<0.05 for single application dextrose and repeated application I-NPs.

FIG. 13 shows an overlay of the total emission of BrP under N₂ (Br; black dashed line) with the Point Grey GS3 camera Red, Green, and Blue channel quantum efficiencies.

FIG. 14 illustrates how Rapid Lifetime Determination (RLD) is computed. The blue line shows a typical decay and the yellow shaded regions represent integrated regions, A1 and A2, bounded by times t1 and t2.

FIG. 15 shows a schematic of the system setup and data processing for real time RLD imaging.

FIG. 16 shows brain imaging using I-NP. Nanoparticles were delivered to the surface of the brain via a murine cranial window that was made through the skull, and ratiometric imaging using ultraviolet (UV) excitation revealed blood vessels in the brain and provided a visual read-out of the amount of oxygen in the brain tissue. Bottom row: Oxygen-sensing nanoparticles were re-applied to the brain 5 minutes after a stroke was surgically initiated, and the ratiometric imaging of the oxygen-sensing nanoparticles revealed a drastic reduction in oxygen levels in the brain tissue, as evidenced by the blue color in the ratiometric image (bottom right panel).

### DETAILED DESCRIPTION

This invention broadly relates to compounds, methods and related imaging systems for oxygen sensing and imaging.

Compounds of the invention are iodo napththyl-phenyl difluoroboron β-diketonate luminescent dye compounds of Formula 1:

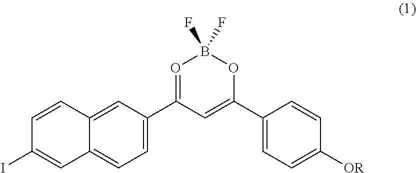

wherein R selected from the group consisting of H, (C₁-C₁₂)alkyl, (C₃-C₁₂)cycloalkyl, (C₁-C₁₀)alkoxy, (C₂-C₁₂)alkenyl, (C₂-C₁₂)alkynyl, (C₁-C₁₂)alkanoyl, (C₁-C₁₂) haloalkyl, (C₁-C₁₂) hydroxyalkyl, (C₁-C₁₂)alkoxycarbonyl, (C₁-C₁₂)alkylthio, (C₂-C₁₂)alkanoyloxy, (C₆-C₂₂)aryl, (C₆-C₁₃)heteroaryl, a polymeric group or combinations thereof. Surprisingly, compounds of Formula 1 exhibit linear oxygen sensitivity (0 to 100%). This full range sensitivity allows for detecting oxygenation levels under normoxic conditions, ambient atmospheric conditions, and beyond. This full range sensitivity is sufficient to distinguish wound and keratinized skin oxygenation, for non-invasive wound diagnosis even without covering the tissue before optical imaging. As shown in Example 3, I-NP of the invention gave consistent measurements of the oxygen levels day to day for covered and uncovered measurements, unlike previously disclose compounds (Examples 4 and 5).

The following abbreviations are used in the description of the invention: nbm(I)OH refers to 1-(4-(2-Hydroxyethoxy)phenyl)-3-(6-iodonaphthalen-2-yl)propane-1,3-dione (aka iodo-napthyl-phenyl β-diketonate). The comparative unsubstituted and bromo analogues are nbmOH and nbm(Br)OH, respectively. BF₂nbm(I)OH refers to nbm(I)OH complexed with difluoroboron. The comparative unsubstituted and bromo analogues are BF₂nbmOH and BF₂nbm(Br)OH, respectively. BF₂nbm(I)PLA or IP refers to the polymer with PLA conjugated to BF₂nbm(I)OH. The comparative unsubstituted and bromo analogues are BF₂nbmPLA or HP and BF₂nbm(Br)PLA or BrP, respectively. I-NP refers to nanoparticles made with BF₂nbm(I)PLA. The comparative unsubstituted and bromo analogues are H-NP and Br-NP, respectively.

The dye portion of the structure of Formula 1 is the portion of the structure except for R. The dye should be in a sufficiently rigid environment so that the triplet excited state decays radiatively (phosphorescence) and does not decay via a non-emissive or non-radiative manner. Providing a rigid environment for the dye is accomplished by directly conjugating the dye with a polymeric group, or dispersing the dye within an additional polymer or other matrix-forming material, or both. Accordingly, polymeric groups or additional polymers with a glass transition temperature greater than the temperature of the surface or environment to be measured are preferred.

In some compounds of the invention, R is H.

For some compounds of the invention, R is selected from the group consisting of (C₁-C₁₂)alkyl, (C₃-C₁₂)cycloalkyl, (C₁-C₁₀)alkoxy, (C₂-C₁₂)alkenyl, (C₂-C₁₂)alkynyl, (C₁-C₁₂)alkanoyl, (C₁-C₁₂) haloalkyl, (C₁-C₁₂) hydroxyalkyl, (C₁-C₁₂)alkoxycarbonyl, (C₁-C₁₂)alkylthio, (C₂-C₁₂)alkanoyloxy, (C₆-C₂₂)aryl, (C₅-C₁₃)heteroaryl, or combinations thereof.

The following definitions are used, unless otherwise described: halo includes fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms. The heteroatoms include non-peroxide oxygen, sulfur, silane, nitrogen and phosphorous wherein suitable substituents as known in the art can be attached to the hetero atoms, for example, hydrogen, O, (C₁-C₁₂)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

For certain compounds of the invention, R is a (C₁-C₁₂)alkyl such as, for example, methyl, ethyl, propyl, isopropyl, butyl, iso-butyl, sec-butyl, pentyl, 3-pentyl, hexyl and the like. For certain compounds of the invention, R is a (C₃-C₁₂)cycloalkyl such as, for example, cyclopropyl, cyclobutyl, cyclopentyl, or cyclohexyl and the like. For certain compounds of the invention, R is a (C₁-C₁₀)alkoxy such as, for example, methoxy, ethoxy, propoxy, isopropoxy, butoxy, iso-butoxy, sec-butoxy, pentoxy, 3-pentoxy, or hexyloxy and the like. For certain compounds of the invention, R is a (C₂-C₁₂)alkenyl such as, for example, vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl and the like. For certain compounds of the invention, R is a (C₂-C₁₂)alkynyl such as, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, A-hexynyl, or 5-hexynyl and the like. For certain compounds of the invention, R is a (C₁-C₁₂)alkanoyl such as, for example, acetyl, propanoyl or butanoyl and the like. For certain compounds of the invention, R is a (C₁-C₁₂) haloalkyl such as, for example, iodomethyl, bromomethyl, chloromethyl, fluoromethyl, trifluoromethyl, 2-chloroethyl, 2-fluoroethyl, 2,2,2-trifluoroethyl, or pentafluoroethyl and the like. For certain compounds of the invention, R is a (C₁-C₁₂) hydroxyalkyl such as, for example, hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl and the like. For certain compounds of the invention, R is a (C₁-C₁₂)alkoxycarbonyl such as, for example, methoxy carbonyl, ethoxy carbonyl, propoxy carbonyl, isopropoxy carbonyl, butoxycarbonyl, pentoxycarbonyl, or hexyloxycarbonyl and the like. For certain compounds of the invention, R is a (C₁-C₁₂)alkylthio can be methylthio, ethylthio, propylthio, isopropylthio, butylthio, isoburylthio, pentylthio, or hexylthio and the like. For certain compounds of the invention, R is a (C₂-C₁₂)alkanoyloxy such as, for example, acetoxy,propanoyloxy, butanoyloxy, isobutanoyloxy, pentanoyloxy, or hexanoyloxy and the like. For certain compounds of the invention, R is a (C₆-C₂₂)aryl such as, for example, phenyl, naphthyl, anthrcyl, phenanthryl, pyryl, naphthacyl, pentacyl, or indenyl and the like. For certain compounds of the invention, R is a (C₅-C₁₃)heteroaryl such as for example, furyl, imidazolyl, triazolyl, triazinyl, oxazoyl, isoxazoyl, tbiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide) and the like.

In certain compounds of the invention, R is a combination of one or more of the above groups and a polymeric group. In a particular compound of the invention, R is a C₂ linked to a polymeric group. A particular compound according to the invention is a compound of Formula 1A:

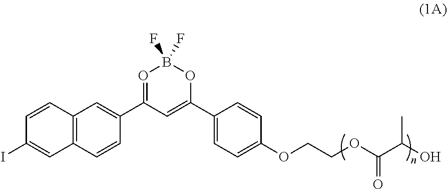

wherein n represents the number of lactide units of the polylactide.

For some compounds of the invention, R is a non-toxic pharmaceutically acceptable, biologically stable (or biodegradable) polymeric group. Non-limiting examples of pharmaceutically acceptable polymeric groups include polylactide (PLA), polyglycolide, lactide-glycolide copolymer, polycaprolactone, or polyethylene glycol polylactide polymers, polyhydroxybutyrate (PHB), polyhydroxybutyrate-valerate copolymer (PHBV), polybutylene succinate (PBS), polybutylene adipate-co-terephthalate (PBAT), sugar based polymers (e.g., cellulose or starch and the like), peptides, nucleic acids, or mixtures thereof. Other exemplary polymeric groups include polyurethanes, polyamides, polyesters, and vinylic polymers. Non-limiting examples of vinylic polymeric groups include acrylates such as polymethyl methacrulate (PMMA), acrylonitrile butadiene styrene (ABS), styrene acrylonitrile (SAN), polystyrenes (PS), polyethylene (PE), polyethylenechlorinates (PEC), polybutadiene (PBD), polydicyclopentadiene (PDCP), polypropylene (PP) Polymethylpentene (PMP), and the like. Other exemplary polymeric groups include silicon-based organic polymers such as polydimethylsiloxane (PDMS), polyesters such as polyethylene terephthalate (PET), glycolized polyester (PETG), polycarbonate (PC) and the like.

Additional exemplary R groups include silica, sol gels, aerogels, xerogels cellulosic polymeric groups, e.g., hydroxypropylmethylcellulose, hydroxyl propyl cellulose, ethyl cellulose and the like; epoxy containing polymeric groups, Ethylene vinyl alcohol, (E/VAL), fluoroplastics, e.g., polytetrafluoroethylene (PTFE), liquid crystal polymeric groups, (LCP), melamine formaldehyde, (MF), phenol-formaldehyde plastic (PF), polyacetal, polyacrylates, polymethacrylates, polyacrylonitrile, (PAN), polyamide, (PA), e.g., nylon, polyamide-imide (PAI), polyaryletherketone (PAEK), polyetheretherketone (PEEK), polyphenylene oxide (PPO), polyphenylene sulfide (PPS), polyphthalamide (PTA), Polysulfone (PSU), polyurethane (PU), polyurea, polyvinylchloride (PVC), polyvinylidene Chloride (PVDC), polyvinylidenedifluoride (PVDF) silicone polymers, poly(ethylene glycol) (PEG), poly(ethylene terephthalate) (PET), polysiloxanes, and silicones.

In certain compounds of the invention, R may be a polylactide, polyglycolide, poly(ethylene glycol), polycaprolactone, lactide-glycolide copolymer, poly(ethylene glycol)-polylactide, polycaprolactone-polylactide, poly(ethylene glycol)-polycaprolactone poly(ethylene glycol)-polylactide-co-glycolide block copolymers, or a mixture thereof. In a particular compound of the invention, R is a polylactide.

In certain compounds of the invention, R is a polymer linked to the rest of the molecule by an alkyl, cycloalkyl, alkoxy, alkenyl, alkynyl, alkanoyl, haloalkyl, hydroxyalkyl, alkoxycarbonyl, alkylthio, alkanoyloxy, aryl, or heteroaryl group. In a particular compound of the invention, R is a polylactide linked to the rest of the molecule through by an C₂ group.

The invention also provides for compositions containing the compound of Formula 1. Some compositions according to the invention comprise the compound of Formula 1 and a solvent. In compositions according to the invention, the solvent is compatible with the surface and the compound, and does not dissolve or cause the degradation of either. In some compositions of the invention, the solvent is water or an aqueous solution. In other compositions, the solvent is an organic solvent. Non-limiting organic solvents include methanol, ethanol, n-propanol, n-butanol, benzyl alcohol, acetone, methyl ethyl ketone, cyclohexanone, chlorobenzene, methyl acetate, n-butyl acetate, dioxane, tetrahydrofuran, methylene chloride and chloroform, and mixtures thereof.

In some compositions of the invention, the compound of Formula 1 is dispersed within an additional polymer or other matrix forming material.

The additional polymer which may be used is, for example, a non-toxic pharmaceutically acceptable, biologically stable (or biodegradable) polymer. Non-limiting examples of pharmaceutically acceptable polymers include polylactide (PLA), polyglycolide, lactide-glycolide copolymer, polycaprolactone, or polyethylene glycol polylactide polymers, polyhydroxybutyrate (PHB), polyhydroxybutyrate-valerate copolymer (PHBV), polybutylene succinate (PBS), polybutylene adipate-co-terephthalate (PBAT), sugar based polymers (e.g., cellulose or starch and the like), peptides, nucleic acids, or mixtures thereof. Other exemplary polymers include polyurethanes, polyamides, polyesters, and vinylic polymers. Non-limiting examples of vinylic polymers include acrylates such as polymethyl methacrulate (PMMA), acrylonitrile butadiene styrene (ABS), styrene acrylonitrile (SAN), polystyrenes (PS), polyethylene (PE), polyethylenechlorinates (PEC), polybutadiene (PBD), polydicyclopentadiene (PDCP), polypropylene (PP) Polymethylpentene (PMP), and the like. Other exemplary polymers include silicon-based organic polymers such as polydimethylsiloxane (PDMS), polyesters such as polyethylene terephthalate (PET), glycolized polyester (PETG), polycarbonate (PC) and the like.

Other additional polymers for the composition of the invention include silica, sol gels, aerogels, xerogels cellulosic polymers, e.g., hydroxypropylmethylcellulose, hydroxyl propyl cellulose, ethyl cellulose and the like; epoxy containing polymers, Ethylene vinyl alcohol, (E/VAL), fluoroplastics, e.g., polytetrafluoroethylene (PTFE), liquid crystal polymers, (LCP), melamine formaldehyde, (MF), phenol-formaldehyde plastic (PF), polyacetal, polyacrylates, polymethacrylates, polyacrylonitrile, (PAN), polyamide, (PA), e.g., nylon, polyamide-imide (PAI), polyaryletherketone (PAEK), polyetheretherketone (PEEK), polyphenylene oxide (PPO), polyphenylene sulfide (PPS), polyphthalamide (PTA), Polysulfone (PSU), polyurethane (PU), polyurea, polyvinylchloride (PVC), polyvinylidene Chloride (PVDC), polyvinylidenedifluoride (PVDF) silicone polymers, poly(ethylene glycol) (PEG), poly(ethylene terephthalate) (PET), polysiloxanes, silicones.

In some compositions of the invention, the compound is dispersed in a non-polymer suitable to stabilize the dye in a rigid environment and still suitable for measuring the optical properties of the dye. In some composition of the invention, the compound is dispersed in a non-polymer matrix such as, for example, a solid composite, ceramic or alloy.

In certain compositions of the invention, the additional polymer may be a polylactide, polyglycolide, poly(ethylene glycol), polycaprolactone, lactide-glycolide copolymer, poly(ethylene glycol)-polylactide, polycaprolactone-polylactide, poly(ethylene glycol)-polycaprolactone poly(ethylene glycol)-polylactide-co-glycolide block copolymers, or a mixture thereof. In a particular composition of the invention, the additional polymer is a polylactide.

The amount of dye relative to the conjugated polymer group in the compound or dispersed within the additional polymer should be low enough allow for a rigid microenvironment to foster radiative decay, but also high enough to be detectable for the imaging techniques used. For example, in some compositions of the invention, the weight to weight ratio or dye : polymer(or polymeric group) ranges from about 1:50 to about 1:300. In some compounds according to the invention, the weight to weight ratio of dye to polymeric group is about 1:200.

Compounds of Formula 1 and compositions of the invention are in the form of powders, films, particles (including e.g., nanoparticles), fibers (including e.g., nanofibers), coatings, bulk materials, gels, networks, assemblies, suspensions, composites, and the like. In a composition according to the invention, a compound of Formula 1 is in the form of a nanoparticle, nanofiber or film. Non-limiting examples of compositions according to the invention include compounds Formula 1 in the form of single or multipolymer nanoparticles or nanofibers in an aqueous suspension; as single or multipolymer nanoparticles or nanofibers embedded in a gel or other polymer or mesh; as a polymeric layer in a multilayer film; or as polymers, nanoparticles, or as nanofibers embedded in a film (for example a sensor foil), gel or composite.

In a preferred embodiment, R is a polylactic acid, and the compound of Formula 1 is in the form of nanoparticles in an aqueous suspension.

Methods according to the invention provide for determining oxygenation levels on a surface comprising the steps of: (a) contacting the surface with compounds of Formula 1, or compositions containing the compounds, under ambient atmospheric conditions; (b) exposing the compound on the surface to an excitation source under ambient atmospheric conditions; (c) detecting the fluorescence and phosphorescence of the compound on the surface under ambient atmospheric conditions; and (d) determining oxygenation levels on the surface based on the ratio of fluorescence to phosphorescence of the compound.

The step of contacting the surface with compounds of Formula 1, or compositions containing the compounds, will vary depending on the surface and on the form of the compounds of Formula 1 or compositions containing the compounds. In methods according to the invention, a compound of Formula 1, or a composition containing the compound, is in the form of a powder, film, particle (including nanoparticle), fiber (including nanofiber), coating, bulk material, gel, suspension, solution, composite or any other suitable form to be placed in contact with the surface. In a method according to the invention, compounds of Formula 1 in the form of a film can be deposited by applying a solution of the compound on the substrate surface, then removing the solvent. In another method according to the invention, a film can be spin-cast onto a surface. Alternatively, a solid form of the compound can be deposited directly onto a surface such as by spraying an aerosol, dust deposition, spreading a melted form, or smearing the solid onto the surface. Various formulations of the compound, can be directly added to the surface as a substrate. In a method according to the invention, the substrate may stay or be a sacrificial layer that can be removed later, for a free standing film. In a method according to the invention, a suspension of the compound formulated as nanoparticles or nanofibers is added dropwise to the surface. Suspensions of nanoparticles or nanofibers applied to biological surfaces may optionally contain pharmaceutical excipients.

In methods according to the invention, the surface may be any desired surface on which oxygenation is to be measured. In some methods according to the invention, the surface is a substrate such as glass, quartz, paper, synthetic paper cloth, plastic sheets, or an inorganic substrate such as ceramics. In some methods according to the invention, the surface is a biological surface, of, for example, living tissues, cells, organisms. In a method according to the invention, the surface is a mammalian tissue such as brain tissue, lung tissue, epithelial tissue, connective tissue, nerve tissue, or muscle or combinations thereof. In a method according to the invention, the surface is a mammalian tissue surface, specifically a wound bed.

In methods according to the invention, the excitation source is a UV lamp, a laser or a LED.

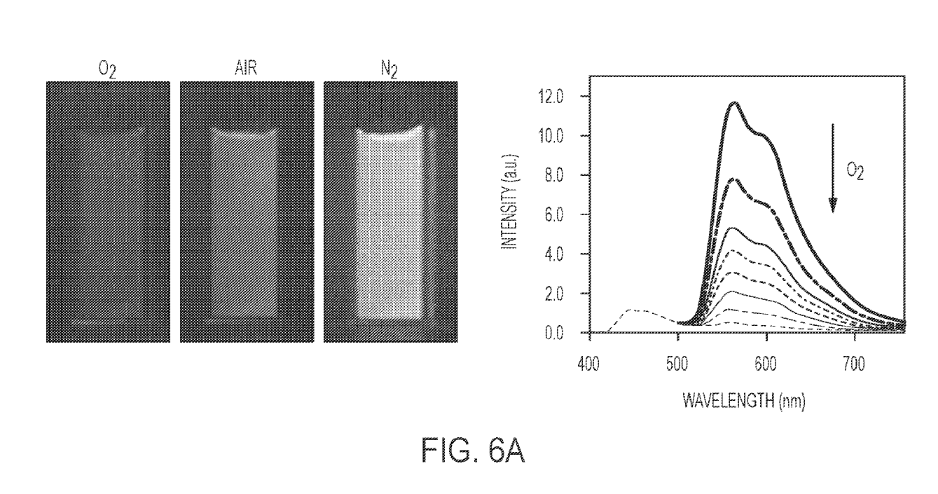

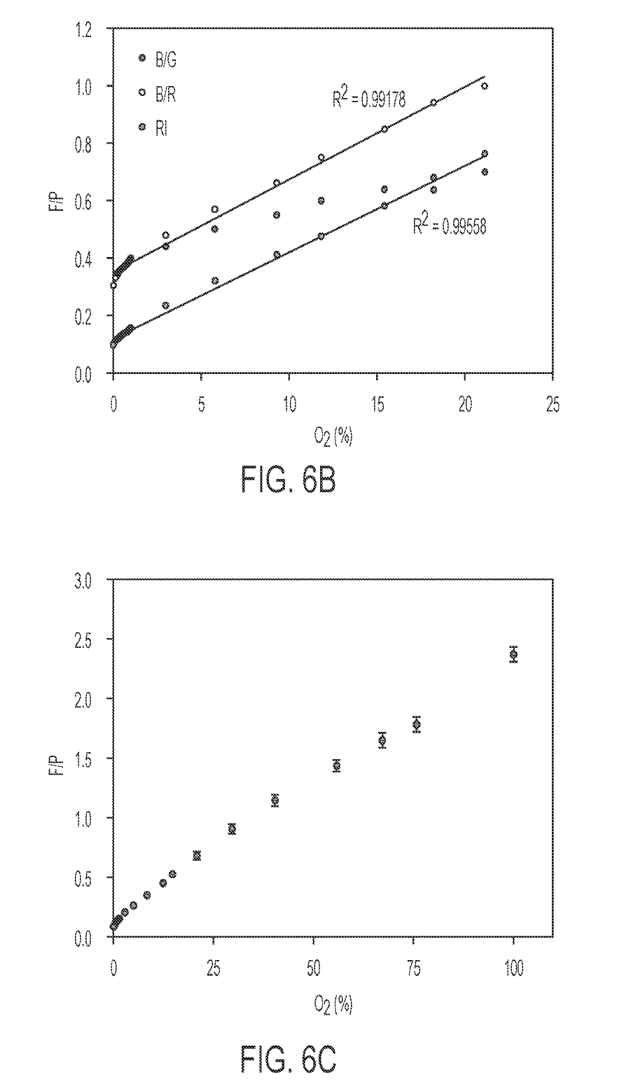

In methods according to the invention, a digital camera is used to detect the fluorescence and phosphorescence of the compound of Formula 1 on a surface. The digital camera may be a digital CCD camera, digital CMOS camera or a digital fluorescence microscopy camera. Red/Green/Blue (RGB) color channels of a digital CCD, CMOS or fluorescence microscopy camera can be used to independently monitor changes in fluorescence and phosphorescence for ratiometric (F/P) sensing. Specific pixels within the area are selected and the intensities of the red and blue color channels at those points are analyzed over the course of the image series. The background color intensities of the images at those points, at times prior to the addition of the compound, are subtracted from the image series for all points within the image. As a result, any subsequent non-zero values for the red and blue channels are the result of the compound's fluorescence (blue channel) and phosphorescence (red channel) only. The ratio of blue light intensity over red light intensity is computed for each pixel to represent the ratio of blue fluorescence (constant in the presence of the compound) to red phosphorescence (quenched in the presence of oxygen). The upper and lower bounds for this ratio are set according to the different sensitivity ranges of the nanoparticle compositions. The ratiometric images are then displayed using a 256-value color map scaled to the ratio bounds for spatiotemporally resolving fluorescence-to-phosphorescence ratios (F/P). As shown in FIG. 6B, for I-NP, the O₂ concentration linearly correlated with both the referenced intensity (RI) which measures the F/P from the fluorescence and phosphorescence maxima peaks from the total emission spectra, as well as B/R, which measures the blue channel to red channel intensity. Depending on the fluorescence and phosphorescence signals, and where they fall in relation to the camera channels, other combinations of channels, such as, for example, green channel/red channel or green channel/blue channel are used.

According to a method of the invention, the compounds of Formula 1, or compositions containing the compounds, and surface are under ambient atmospheric conditions. Under these conditions, the compounds of Formula 1, or compositions containing the compounds, on the surface is not isolated from ambient oxygen in air. In alternative methods of the invention, after the compounds of Formula 1, or compositions containing the compounds, is contacted with the surface, the surface is covered to slow or block the surface from ambient oxygen in air. In another method of the invention, the compounds of Formula 1, or compositions containing the compounds, and surface are under conditions where the oxygen concentration is greater than atmospheric oxygen concentration.

In some methods of the invention where a suspension of nanoparticles, nanofibers, or other compositions containing compounds of Formula 1, are applied to a surface, the volume of the suspension is small enough to just cover the surface. For example, a volume of 10 μL of an aqueous suspension of I-NP is added to a 3 mm wound bed.

In a method according to the invention, an aqueous suspension of I-NP is applied to brain tissue; exposed to an excitation source, and the fluorescence and phosphorescence are detected, all under ambient atmospheric conditions, and the oxygenation levels at the surface are determined based on the ratio of fluorescence to phosphorescence with a digital camera, where the ratio of fluorescence to phosphorescence is measured as the relative intensity of the blue channel to red channel.

In a method according to the invention, an aqueous suspension of I-NP is applied to a wound bed; exposed to an excitation source, and the fluorescence and phosphorescence are detected, all under ambient atmospheric conditions, and the oxygenation levels at the surface are determined based on the ratio of fluorescence to phosphorescence with a digital camera, where the ratio of fluorescence to phosphorescence is measured as the relative intensity of the blue channel to red channel.

Methods according to the invention provide for monitoring wound healing over one or more days, by determining the oxygenation levels on the uncovered wound, by contacting the uncovered wound with compounds of Formula 1, or compositions containing the compounds, exposing the compound on the uncovered wound to an excitation source; detecting the fluorescence and phosphorescence of the compound on the uncovered wound; and determining oxygenation of the wound based on the ratio of fluorescence to phosphorescence of the compound.

In methods for monitoring wound healing according to the invention, compounds of Formula 1, or compositions containing the compounds, are directly added to the wound. For example, a suspension of the compound in the form of nanoparticles or nanofibers is added dropwise to the surface. Suspensions of nanoparticles or nanofibers applied to wounds may optionally contain pharmaceutical excipients. In some methods of the invention where a suspension of nanoparticles, nanofibers, or other compositions containing compounds of Formula 1, are applied to the wound, the volume of the suspension is small enough to just cover the wound. For example, a volume of 10 μL of an aqueous suspension I-NP is added to a 3 mm wound bed.

In methods for monitoring wound healing according to the invention, the excitation source is a UV lamp, laser or a LED.

In a method for monitoring wound healing according to the invention, a digital CCD, CMOS or fluorescence microscopy camera is used to detect the fluorescence and phosphorescence of the compound of Formula 1 on a wound. Red/Green/Blue (RGB) color channels of a digital CCD, CMOS or fluorescence microscopy camera can be used to independently monitor fluorescence (blue channel) and phosphorescence (red channel) for ratiometric (F/P) sensing. Specific pixels within the area are selected and the intensities of the red and blue color channels at those points are analyzed over the course of the image series. The background color intensities of the images at those points, at times prior to the addition of the compound, are subtracted from the image series for all points within the image. As a result, any subsequent non-zero values for the red and blue channels are the result of compound's fluorescence and phosphorescence only. The ratio of blue light intensity over red light intensity is computed for each pixel to represent the ratio of blue fluorescence (constant in the presence of NPs) to red phosphorescence (quenched in the presence of oxygen). The upper and lower bounds for this ratio are set according to the different sensitivity ranges. The ratiometric images are then displayed using a 256-value color map scaled to the ratio bounds for spatiotemporally resolving fluorescence-to-phosphorescence ratios (F/P). Depending on the fluorescence and phosphorescence signals, and where they fall in relation to the camera channels, other combinations of channels, such as, for example, green channel/red channel or green channel/blue channel are used.

In a method for monitoring wound healing according to the invention, the wound is uncovered. Under these conditions, the compounds of Formula 1, or compositions containing the compounds, on the wound are not isolated from ambient oxygen in air. In alternative methods, after the compounds of Formula 1, or compositions containing the compounds, are applied to the wound, the wound is covered to slow or block the surface from ambient oxygen in air. For example, a glass coverslip is added on top of the wound prior to the excitation and detection steps. In other methods, nanoparticle comprising the compound of Formula 1 are applied or embedded in a cover, or the sensing material itself is a cover.

In a method for monitoring wound healing according to the invention, compounds of Formula 1, or compositions containing the compounds, are applied once, and only the excitation and detection steps are performed daily. In a method for monitoring wound healing according to the invention compounds of Formula 1, or compositions containing the compounds, are applied to the wound daily, and the excitation and detection steps are performed subsequently.

In a method for monitoring wound healing according to the invention, an aqueous suspension of I-NP is applied to an uncovered wound daily; exposed to an excitation source, and the fluorescence and phosphorescence are detected, all under ambient atmospheric conditions, and oxygenation levels at the surface are determined based on the ratio of fluorescence to phosphorescence with a digital camera, where the ratio of fluorescence to phosphorescence is measured as the relative intensity of the blue channel to red channel.

The imaging system according to the invention comprises a compound of Formula 1, and excitation source such as a UV lamp, laser or LED, a digital camera, and a computer for data processing. A digital CCD or CMOS camera is an ideal tool for two-dimensional analysis. At its core is an array of photosensors (pixels) that convert incident photons into a digital signal. Each pixel acts as its own sensing element providing spatial resolution, while a gated shutter provides temporal resolution. These processes are all performed on-board by the digital chip allowing for simple operation.

The invention also provides methods for determining oxygenation on a surface comprising the steps of contacting the surface with compounds of Formula 1, or compositions containing the compounds, exposing the compound on the surface to an excitation source, detecting the room temperature phosphorescence lifetime of the compound, and determining the oxygenation levels on the surface based on the phosphorescence lifetime. In a method according to the invention, phosphorescence lifetimes are measured by monitoring the luminescent compound of Formula 1 on a surface with a digital camera, as previously described.⁴⁹

### EXAMPLES

### Example 1

BF₂nbm(X)PLA polymers were synthesized and their optical properties were measured.

Materials and Methods

3,6-Dimethyl-1,4-dioxane-2,5-dione (D,L-lactide, Sigma Aldrich) was recrystallized twice from ethyl acetate and stored under nitrogen. The ligand precursors, methyl 6-iodo-2-naphthoate,⁹³ and 1-(4-(2-((tetrahydro-2H-pyran-2-yl)oxy)ethoxy)phenyl)ethan-1-one,⁹⁴ and boron initiators BF₂nbmOH⁸⁸ and BF₂nbm(Br)OH³⁶ were prepared as previously described. Polymers were prepared by ring-opening polymerization from racemic lactide by a method previously described. Tin(II) 2-ethylhexanoate (Sn(oct)₂, Spectrum), boron trifluoride diethyl etherate (Aldrich, purified, redistilled), and all other reagents and solvents were used as received without further purification. Solvents CH₂Cl₂ and THF were dried and purified over 3 Å molecular sieves activated at 300° C.⁹⁵ All other chemicals were reagent grade from Sigma-Aldrich and were used without further purification. ¹H NMR spectra were recorded on a Varian VMRS/600 (600 MHz) instrument in CDCl₃. ¹H NMR peaks were referenced to the signals for the residual protiochloroform at 7.26 ppm. Coupling constants are given in hertz. Polymer molecular weights were determined by gel permeation chromatography (GPC) (THF, 25° C., 1.0 mL/min, dn/dc=0.050) using multiangle laser light scattering (SEC-MALS) (λ=658 nm, 25° C.) and refractive index (RI) (λ=658 nm, 25° C.) detection. Polymer Laboratories 5 μm mixed-C columns (guard column plus two columns) along with Wyatt Technology (Optilab T-rEX interferometric refractometer, miniDAWN TREOS multiangle static light scattering (MALS) detector, ASTRA 6.0 software) and Agilent Technologies instrumentation (series 1260 HPLC with diode array (DAD) detector, ChemStation) were used in GPC analysis. UV/vis spectra were recorded on a Hewlett-Packard 8452A diode-array spectrophotometer. The CMOS camera (PGR GS3-U3-41C6C-C) and image processing were performed as previously described. For dual mode imaging, Br-NPs were continuously illuminated with a handheld UVP UV lamp (λₑₓ=365 nm), then lifetime and ratiometric detection were performed as previously described.⁴⁹

RLD lifetime measurements. Samples are placed approximately 0.5 m below the camera and are excited by a manually triggered Yongnou 560-II flash unit masked by an Esco Optics 425 nm bandpass filter (40 nm bandwidth). Pulses with reproducible profiles and durations as short as 50 μs can be generated at regular intervals for imaging. Images are captured with a PGR GS3-U3-41C6C-C video camera equipped with a Spacecom f/0.95 50 mm lens and an Edmund Optics 425 nm long pass filter to minimize excitation background. The camera has a color CMOS chip capable of 90 frames per second (FPS) at a maximum resolution of 2048×2048 pixels. Framerates up to 2000 FPS can be achieved at reduced resolutions. Camera data and power are provided through a USB 3.0 cable connected to a Lenovo w530 laptop, which is responsible for camera control, data acquisition, processing, and display.

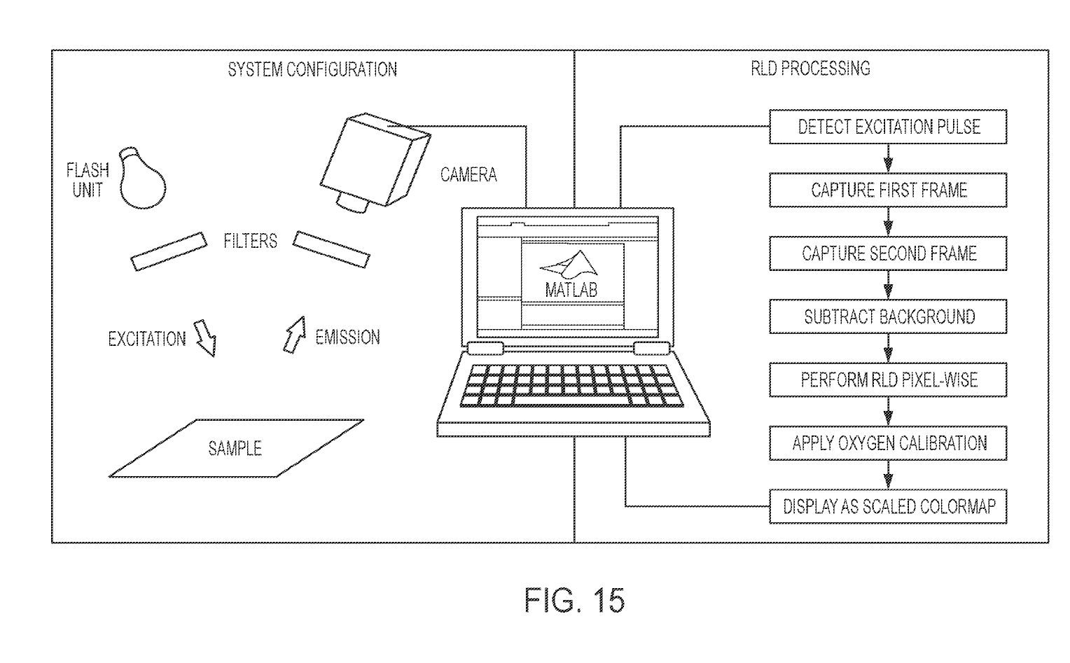

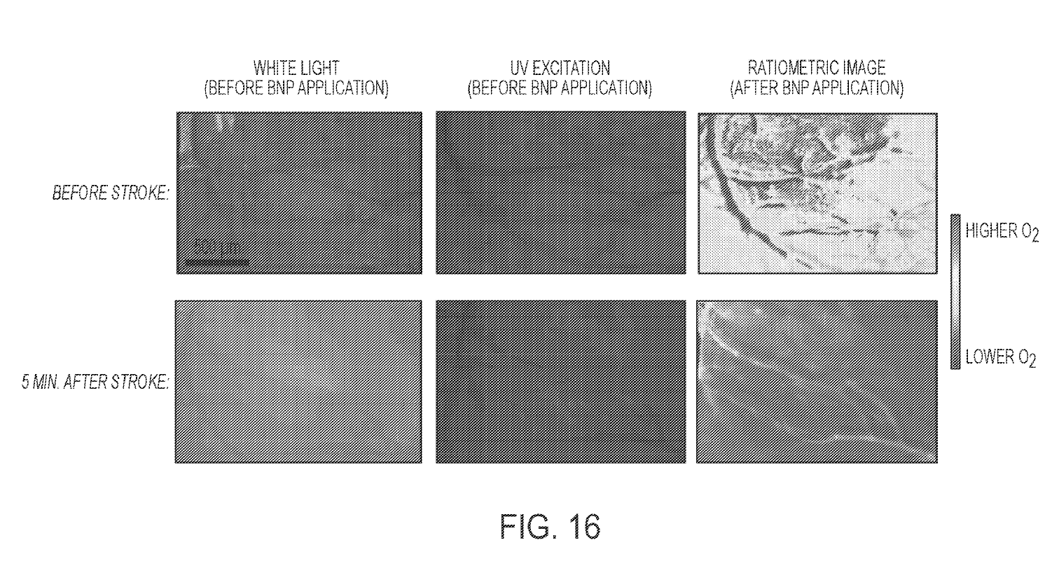

RLD software design. The camera records 8-bit Bayer data and performs a nearest-neighbor demosaicing algorithm on-board unless otherwise specified. Camera control, data acquisition, processing, and display are all performed in custom MATLAB 2014b programs. The Image Acquisition and Curve Fitting toolboxes are necessary add-ons for these programs. The apparent light intensity is controlled primarily through the shutter speed. Gain may be increased if the image is underexposed. Otherwise it is turned off. Gamma correction is always set to a value of 1 (meaning no additional amplification or distortion of the sensor output is applied). The white balancing feature is also turned off. The region of interest (ROI) may be specified to reduce data output or increase framerate. The FPS is set based on the calculation method (NLS vs RLD) as well as the range of expected lifetimes. Total intensity at a single pixel is determined by summing the 8-bit values from red, green, and blue channels. If single, absolute lifetime measurements are desired, the NLS method is used and the FPS is set such that at least ten frames will be acquired during the decay. The beginning of the decay is detected in software by the appearance of the excitation peak. After monitoring the decay, all pixel intensities in the ROI are averaged frame-by-frame and fit as a function of time (determined by the FPS) to a single or multiexponential decay function with an offset. In the case of a single exponential decay, the lifetime may be extracted directly from the fit parameters. For a multiexponential decay, a weighted lifetime is calculated using pre-exponential weighting.⁸⁷ Oxygen imaging may be performed by computing the lifetime at each pixel, applying a predetermined oxygen calibration, and displaying the resultant distribution as a scaled colormap. Continuous lifetime measurements were performed by RLD imaging. FIG. 15 shows the schematic for data processing real time RLD imaging. The frames per second (FPS) is set such that at least two frames will be acquired during the decay. Because an offset can significantly affect the ratio of the denominator of the RLD equation, a background image is captured before imaging begins. The excitation pulse is detected in software and at least two consecutive frames are subsequently captured. The background image is subtracted pixel-wise from each frame. The lifetime at each pixel is then determined by Equation in FIG. 14 where the numerator is the inverse framerate and the denominator is given by the natural logarithm of the ratio of intensities. A predetermined oxygen calibration is then applied to each pixel to determine the concentration. The oxygen distribution is displayed as a scaled colormap. The program then awaits the next excitation pulse. Because the decay times are substantially longer than the RLD processing time, real time processing may be performed at full resolution at 2 FPS. If lifetimes are long enough to capture more than two frames, the width and timing of the integration intervals may be changed in software for optimal performance.

Luminescence Measurements. Steady-state fluorescence emission spectra were recorded on a Horiba Fluorolog-3 Model FL3-22 spectrofluorometer (double-grating excitation and double-grating emission monochromator). A 1 ms delay was used when recording the delayed emission spectra. Time-correlated single-photon counting (TCSPC) fluorescence lifetime measurements were performed with a NanoLED-370 (λₑₓ=369 nm) excitation source and a DataStation Hub as the SPC controller. Phosphorescence lifetimes were measured with a 1 ms multichannel scalar (MCS) excited with a flash xenon lamp (λₑₓ=369 nm; duration <1 ms). Lifetime data were analyzed with DataStation v2.4 software from Horiba Jobin Yvon. Thin films were prepared on the inner wall of vials by dissolving polymers in CH₂Cl₂ (2 mg/mL) and evaporating the solvent by slowly rotating the vial under a low stream of nitrogen. The solution-cast films were then dried in vacuo overnight before measurements. Fluorescence spectra and lifetimes of the films were obtained under ambient conditions (e.g., air, ˜21% oxygen). The vials with the solution-cast films were purged and sealed with a Teflon cap and wrapped in parafilm in a glove box prior to phosphorescence measurements. The glove box was purged for 30 min prior to samples being sealed. Oxygen calibration of the nanoparticles was done in triplicate as previously described using analytical grade gases (Cole-Palmer flow gauges equipped with a mixing chamber; Praxair: pure N₂, 1.0% O₂, 21.0% O₂, or 100% O₂).²⁷ Fluorescence and phosphorescence lifetimes were fit to double exponential decays. Spin-cast films and photostability measurements were done as previously described.³⁸

Synthesis. 1-(4-(2-Hydroxyethoxy)phenyl)-3-(6-iodonaphtha-len-2-yl)propane-1,3-dione (nbm(I)OH). The aromatic ketone 1-{4-[2-(tetrahydropyran-2-yloxy)-ethoxy]-phenyl}-ethanone (500 mg, 1.89 mmol) and 6-iodo, 2-methyl naphthoate (710 mg, 2.27 mmol) were added to a 250 mL oven dried round bottom flask and dissolved in anhydrous THF (˜100 mL). A suspension of NaH (91 mg, 3.87 mmol) in THF (20 mL) was transferred to the reaction via cannula. The reaction was refluxed at 60° C. in a nitrogen atmosphere, and monitored by TLC. Upon consumption of the ketone limiting reagent (14 h), the reaction mixture was removed from the oil bath and allowed to cool to RT. Excess NaH was quenched with sat. NaHCO₃ (20 mL), and solvents were removed via rotary evaporation. The pH was fixed to ˜5 with 1M HCl and the mixture was extracted with CH₂Cl₂ (20 mL×2) and washed with H₂O (20 mL×2), and brine (20 mL×2). Crude product was passed through a silica plug with CH₂Cl₂ before dissolution in THF/H₂O (40 mL/10 mL). A catalytic amount of TsOH (25 mg, 0.15 mmol) was added the reaction mixture was refluxed at 60° C. in a nitrogen atmosphere (12 h). Solvents were concentrated via rotary evaporation and the product was extracted with CH₂Cl₂ (100 mL×3), and washed with H₂O (20 mL×2), and brine (20 mL×2). The organic layer was dried over anhydrous Na₂SO₄, filtered and solvents were removed via rotary evaporation. Crude product was purified by recrystallization with acetone/hexanes to yield a white powder: 326 mg (37%). ¹H NMR (600 MHz, D₆-DMSO): δ17.37 (s, 1H, enol-OH), 8.77 (s, 1H, 1′-ArH), 8.49 (s, 1H, 5′-ArH), 8.20 (d, J=12, 1H, 8′-ArH) 8.16 (d, J =12, 2H, 2″, 6″-ArH), 7.99 (d, J=12, 1H, 7′-ArH), 7.88 (s, broad, 2H, 3′, 4′-ArH), 7.40 (s, 1H, COCHCO), 7.10 (d, J=12, 2H, 3″, 5″-ArH), 4.90 (t, J=6, 1H, Ar—OCH₂CH₂OH), 4.10 (t, J=6, 2H, Ar—OCH₂CH₂OH), 3.73 (m, broad, 2H, Ar—OCH₂CH₂OH). HRMS (ESI, TOF) m/z calcd for C₂₁H₁₈O₄I, 461.0250 [M+H]⁺; found 461.0250.

BF₂nbm(I)OH. The iodide dye was prepared by weighing ligand, nbm(I)OH (150 mg, 0.32 mmol), in a 250 mL round bottom flask and dissolving in anhydrous THF (150 mL). Boron trifluoride diethyl etherate (61 μL, 0.50 mmol) was added via syringe and the solution turned yellow. The reaction mixture was refluxed at 60° C. under a N₂ atmosphere and monitored by TLC until consumption of the ligand substrate was complete (2 h). Excess boron trifluoride diethyl etherate was quenched with K₂CO₃₍ₛ₎ (˜30 mg) and stirred for an additional 15 min. The solution was filtered to remove solids, and solvents were removed via rotary evaporation to yield a dark yellow powder. The product was purified by recrystallization (acetone/hexanes) to yield a yellow powder: 105 mg (62%). ¹H NMR (600 MHz, D6-DMSO): δ9.02 (s, 1H, 1′-ArH), 8.55 (s, 1H, 5′-ArH), 8.40 (d, J=12, 2H, 2″, 6″-ArH), 8.35 (d, J=12, 1H, 8′-ArH), 8.06 (d, J=12, 1H, 7′-ArH), 7.97-7.93 (m, broad, 3H, 3′, 4′-ArH, COCHCO), 7.22 (d, J=12, 2H, 3″, 5″-ArH), 4.94 (t, J=6, 1H, Ar—OCH₂CH₂OH), 4.18 (t, J=6, 2H, Ar—OCH₂CH₂OH), 3.75 (m, broad, 2H, Ar—OCH₂CH₂OH). HRMS (ESI, TOF) m/z calcd for C₂₁H₁₈BO₄F₂I, 508.0154 [M+H]⁺; found 508.0149.

BF₂nbmPLA (HP). The unsubstituted polymer (HP) was prepared as previously described³⁷ (loading=initiator:lactide:catalyst; 1:200:0.025) to yield a yellow/white crystalline powder: 805 mg (66% yield, corrected for 82% polymer conversion). Mₙ(GPC/MALS)=19 900 Da, D=1.12; M_{w} (¹H NMR)=20 300 Da. ¹H NMR (600 MHz, CDCl₃): δ 8.76 (s, 1H, 1′-ArH), 8.20 (d, J=12, 2H, 2″, 6″-ArH), 8.08 (d, J=12, 1H, 8′-ArH), 8.01 (d, J=6, 1H, 5′-ArH), 7.96 (d, J=6, 1H, 3′-ArH), 7.91 (d, J=12, 1H, 4′-ArH) 7.66 (t, J=6, 1H, 7′-ArH), 7.60 (t, J=6, 1H, 6′-ArH), 7.28 (s, 1H, COCHCO), 7.06 (d, J=12, 2H, 3″, 5″-ArH), 5.23-5.12 (m, broad, 282H, PLA-H), 4.55 (s, broad, 2H, Ar—OCH₂CH₂OH), 4.32 (m, broad, 2H, Ar—OCH₂CH₂OH), 1.58-1.53 (m, broad, 911H, PLA-CH₃).

BF₂nbm(Br)PLA (BrP). The bromide substituted polymer was prepared as previously described³⁷ (loading=initiator:lactide:catalyst; 1:200:0.025) by to yield a yellow crystalline powder: 520 mg (78% yield, corrected for 72% polymer conversion). Mₙ(GPC/MALS)=16 400 Da, D=1.10; M_{w} (¹H NMR)=22 100 Da. ¹H NMR (600 MHz, CDCl₃): δ 8.79 (s, 1H, 1′-ArH) 8.20 (d, J=6, 2H, 2″, 6″-ArH), 8.10 (m, broad, 2H, 5′, 8′-ArH), 7.88 (m, 2H, 3′, 7′-ArH) 7.68 (d, J=6, 1H, 4′-ArH), 7.07 (s, 1H, COCHCO), 5.23-5.12 (m, broad, 307H, PLA-H), 4.55 (s, broad, 2H, Ar—OCH₂CH₂OH), 4.32 (m, broad, 2H, Ar—OCH₂CH₂OH), 1.58-1.53 (m, broad, 1121H, PLA-CH₃).

BF₂nbm(I)PLA (IP) The iodide substituted polymer was prepared as previously described,³⁷ except the initiator BF₂nbm(I)OH was used in place of BF₂nbm(Br)OH (loading=initiator:lactide:catalyst; 1:200:0.025), and was stirred at 130° C. for 5 h, to yield a yellow crystalline powder: 345 mg (51% yield, corrected for 65% polymer conversion). Mₙ(GPC/MALS)=16 300 Da, D=1.17; M_{w} (¹H NMR)=19 300 Da. ¹H NMR (600 MHz, CDCl₃): δ8.71 (s, 1H, 1′-ArH), 8.33 (s, 1H, 5′-ArH), 8.20 (d, J=6, 2H, 2″, 6″-ArH), 8.09 (d, J=6, 1H, 8′-ArH), 7.85 (m, broad, 2H, 3′, 4′-ArH) 7.73 (d, J=12, 1H, 7′-ArH), 7.16 (s, 1H, COCHCO), 7.04 (d, J=12, 2H, 3″, 5″-ArH), 5.23-5.12 (m, broad, 268H, PLA-H), 4.55 (s, broad, 2H, Ar—OCH₂CH₂OH), 4.32 (m, broad, 2H, Ar—OCH₂CH₂OH), 1.58-1.53 (m, broad, 1137H, PLA-CH₃).

Table 1 summarizes the optical properties of the boron dye initiators and polymers in CH₂Cl₂.

TABLE 1
Optical Properties of Boron Dye Initiators and Polymers in CH₂Cl₂

| | | λ_{abs}^{a} | ε^{b} | λₑₘ^{c} | τ_{F}^{d} | |
|---|---|---|---|---|---|---|
| Sample | | (nm) | (M⁻¹ cm⁻¹) | (nm) | (ns) | φ_{F}^{e} |
| BF₂nbmOH | | 414^{f} | 59 000^{f} | 452^{f} | 1.55^{f} | 0.40^{f} |
| BF₂nbmPLA | HP | 414 | 53 200 | 456 | 1.54 | 0.40 |
| BF₂nbm(Br)OH | | 417^{f} | 65 000^{f} | 448^{f} | 0.53^{f} | 0.19^{f} |
| BF₂nbm(Br)PLA | BrP | 417 | 60 400 | 448 | 0.49 | 0.20 |
| BF₂nbm(I)OH | | 419 | 66 100 | 444 | 0.20 | 0.05 |
| BF₂nbm(I)PLA | IP | 419 | 62 500 | 445 | 0.20 | 0.05 |

^{a}Absorption maxima.
^{b}Extinction coefficients calculated at the absorption maxima.
^{c}Fluorescence emission maxima excited at 369 nm.
^{d}Fluorescence lifetime excited with a 369 nm light-emitting diode (LED) monitored at the emission maximum. All fluorescence lifetimes are fitted with single-exponential decay.
^{e}Relative quantum yield, versus anthracene in EtOH as a standard.¹⁴
^{f}Values taken from Samonina-Kosicka et al. Macromolecules, 2014, 47, 3736-3746.³⁶

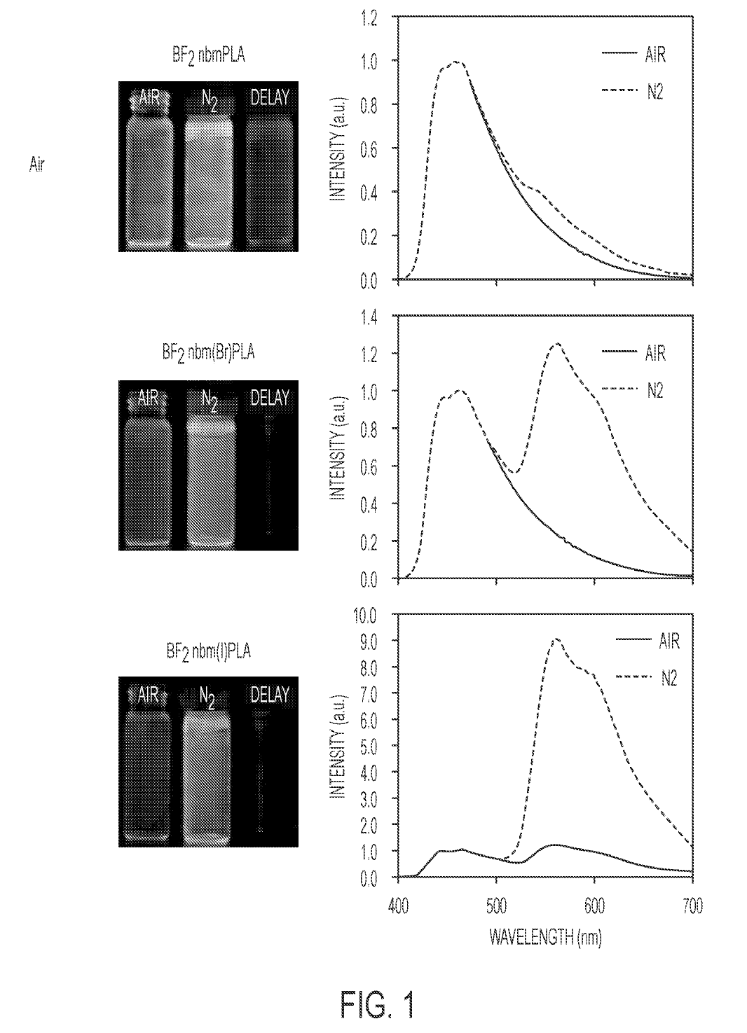

FIG. 1 shows the optical properties of the polymer films. Photographs of dye-polymers under air, N₂, and under N₂ with the lamp turned off (delay) and corresponding total emission spectra in air and N₂ (λₑₓ=385 nm).

Table 2 summarizes the optical properties of the polymer films.

TABLE 2
Optical Properties of Polymer Films

| | | | | |
|---|---|---|---|---|
| | λ_{F}^{a} | τ_{F}^{b} | λ_{P}^{c} | τ_{P}^{d} |
| Sample | (nm) | (ns) | (nm) | (ms) |

| | | | | | |
|---|---|---|---|---|---|
| BF₂nbmPLA | HP | 459 | 1.76 | 545 | 453 |
| BF₂nbm(Br)PLA | BrP | 462 | 0.86 | 561 | 14.5 |
| BF₂nbm(I)PLA | IP | 461 | 0.47 | 569 | 1.90 |

^{a}Steady-state fluorescence emission maximum (λₑₓ = 385 nm)
^{b}Fluorescence lifetime (λ_{Ex} = 369 nm LED)
^{c}Delayed emission spectra maxima under N₂ (λₑₓ = 385 nm)
^{d}Pre-exponential weighted RTP lifetime.

The polymer luminescence was analyzed as thin films in glass vials (Table 1 and FIG. 1). Additionally, dye initiators and dye-PLA conjugates had very similar properties in dilute CH₂Cl₂ solutions, indicating that the integrity of the dye is maintained during the polymerization process (i.e. negligible boron decomplexation).^{41,42}

For solid state films, all polymers have indistinguishable blue fluorescence at ˜460 nm and lifetimes, τ_{F}<2 ns. Phosphorescence red-shifted and the RTP intensity increased relative to the fluorescence, while lifetimes decreased more dramatically, as is expected for the HP to BrP and IP series given the heavy atom effect.⁴³ A weak phosphorescence shoulder and long lifetime (τ_{P}=453 ms) were observed for HP. The bromide polymer, BrP, showed two distinguishable peaks for fluorescence and phosphorescence, and a decreased lifetime (14.5 ms). Whereas, phosphorescence dominated for the iodide polymer (IP), and the lifetime further shortened (1.9 ms). These results show that halide substitution primarily influenced the RTP, while features of the fluorescence (e.g. color) are well maintained. Because changes in color are negligible, detection methods can be broadly applied without changing settings (e.g. filters), and the three materials can be easily interchanged to screen and identify the optimal material for a given sensing application.

Oxygen Sensing. The halide substituted dye-PLA conjugates are distinguished by their RTP intensities and unquenched lifetimes, which relate to two ways to quantify oxygen quenching (l₀/l or τ₀/τ).^{7,44} As shown in equations 1 and 2, the unquenched RTP lifetime (τ₀) is directly correlated to the Stern-Volmer quenching constant (KSV).

$$\begin{matrix}\frac{{I}_{0}}{I} = \frac{{τ}_{0}}{τ} = 1 + {K}_{SV} [ Q ] & ( 1 ) \\ {K}_{SV} = {k}_{q} {τ}_{0} & ( 2 )\end{matrix}$$

As a result, materials with long RTP lifetimes are more sensitive to oxygen (Q) quenching (large KSV), and will operate within a narrower O2 sensing range. Furthermore, materials with longer lifetimes can be detected with less costly instrumentation (e.g. lower frame rate).

Halide activated RTP influences the oxygen sensitivity and mode of detection via lifetime (τ/τ₀) or intensity (l/l₀) techniques. The hydrogen substituted dye-polymer (HP), with weak RTP and a long lifetime (˜400 ms) serves as an ultrasensitive lifetime oxygen sensor, whereas the iodide derivative (IP), with a short lifetime (˜2 ms) but intense RTP, functions as a full range ratiometric sensor. The bromide derivative showed balanced F and RTP intensities, and a relatively long RTP lifetime (˜14 ms). Therefore, with BrP, oxygen can be sensed via both detection modes.

### Example 2

To elucidate the oxygen sensing ranges and generate materials suitable for wound application, the polymers were fabricated as nanoparticles³² (X-NPs, where X═H, Br, and I) and subjected to oxygen calibration²⁷

Nanoparticle Fabrication. Nanoparticles (˜1 mg/mL) were prepared as previously described by DMF/H₂O precipitation into deionized water.³² Cellular isotonic conditions were achieved by the addition of dextrose to yield a 5% dextrose/NP/H₂O solution. The NP solution (˜6 mL of ˜1 mg/mL) was concentrated by centrifugation at 4000 rpm (room temperature) for 3 min (Sorval, ThermoScientific, Legend RT) in a concentrator centrifuge tube (Amicon Ultra, Regenerated Celluose, 30,000 MW cutoff) to yield ˜3 mL of a ˜2 mg/mL NP solution. To remove aggregates, ˜2 mg/mL NP solution (1 mL) was passed through a 200 nm filter (Whatman). Then 10% dextrose solution (1 mL) was added to yield 2 mL of ˜1 mg/mL solution at 5% dextrose concentration. The NP solutions were stored at 5° C. prior to use, and were filtered (200 nm Whatman) to sterilize just prior to wound application.

Table 3 shows the optical properties of nanoparticles.

TABLE 3
Optical Properties of Nanoparticles

| | DLS^{a} | | Fluorescence | | Phosphorescence | | |
|---|---|---|---|---|---|---|---|
| | R_{H} | | λ_{F}^{b} | τ_{F}^{c} | λ_{P}^{d} | τ_{P}^{e} | τ₀/ |
| NP | (nm) | PD | (nm) | (ns) | (nm) | (ms) | τ_{1%}^{f} |
| H | 38.5 | 0.09 | 459 | 1.76 | 543 | 127 | 32.0 |
| Br | 37.0 | 0.11 | 462 | 0.86 | 559 | 12.3 | 3.3 |
| I | 41.4 | 0.18 | 461 | 0.47 | 565 | 1.7 | 0.8 |

^{a}NP hydrodynamic radius (R_{H}) and polydispersity (PD) determined by dynamic light scattering (DLS).
^{b}Steady-state fluorescence spectra emission maximum under air. Excitation source: monochromator set to 385 nm with xenon lamp.
^{c}Fluorescence lifetime excited with a 369 nm light-emitting diode (LED) monitored at the emission maximum.
^{d}Delayed emission spectra maxima under N₂. Excitation source: monochromator set to 385 nm with xenon lamp.
^{e}Pre-exponential weighted RTP lifetime.
^{f}Sensitivity measurement of NPs.⁷

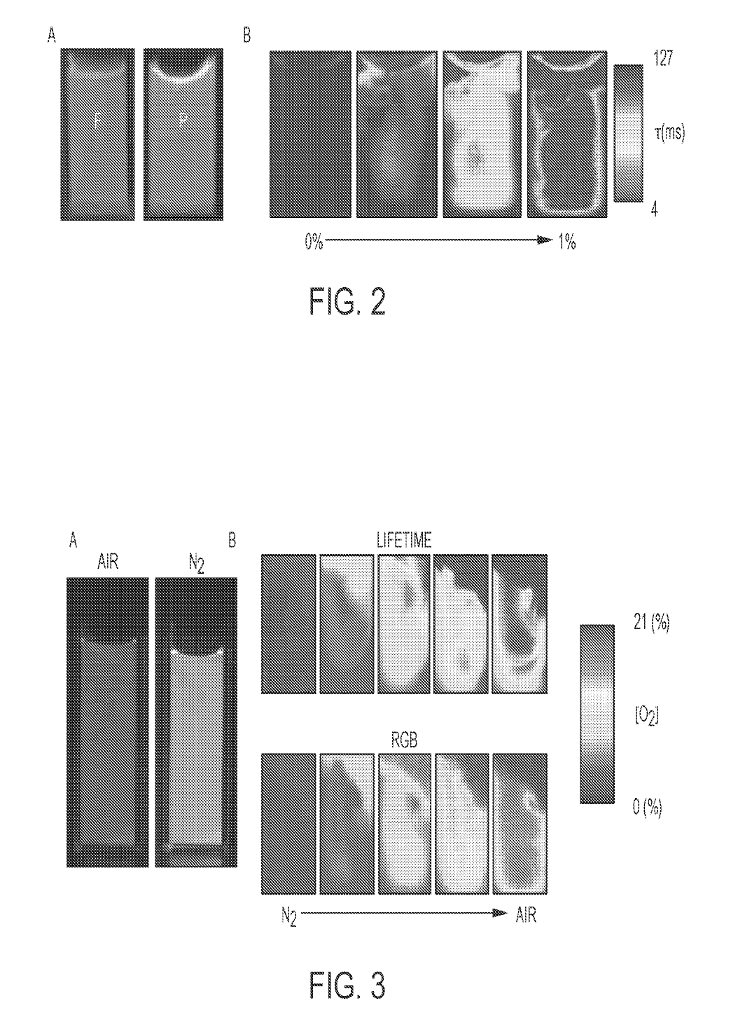

For the H-NP, as shown in FIG. 2, phosphorescence lifetime vs [O₂] showed an oxygen sensing range of approximately 0-0.3%. The long-lived RTP of H-NP was quenched very quickly (i.e. in ˜6 s) when 1% O₂ was bubbled into a nitrogen purged sample.

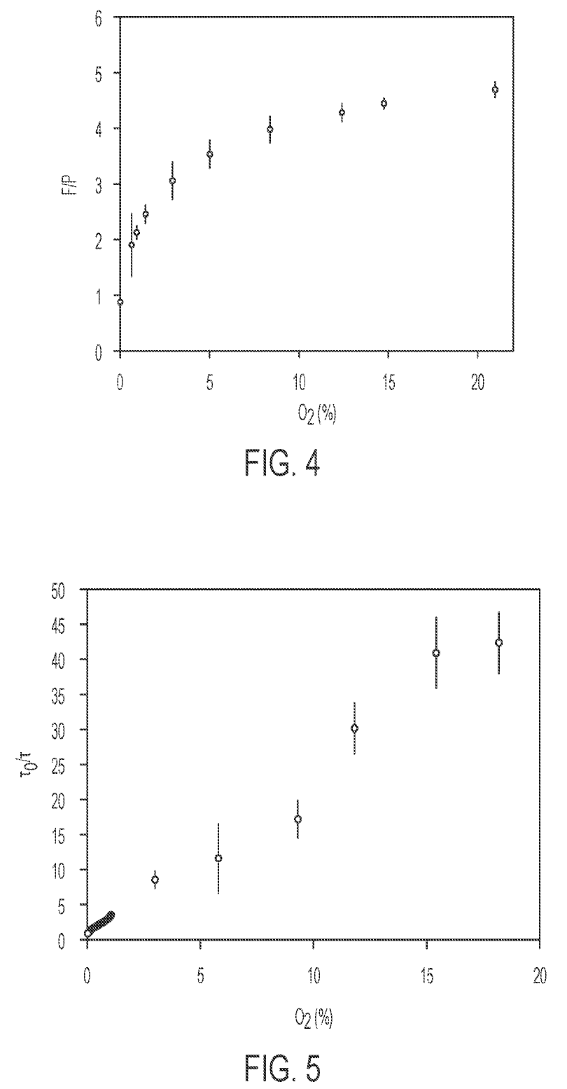

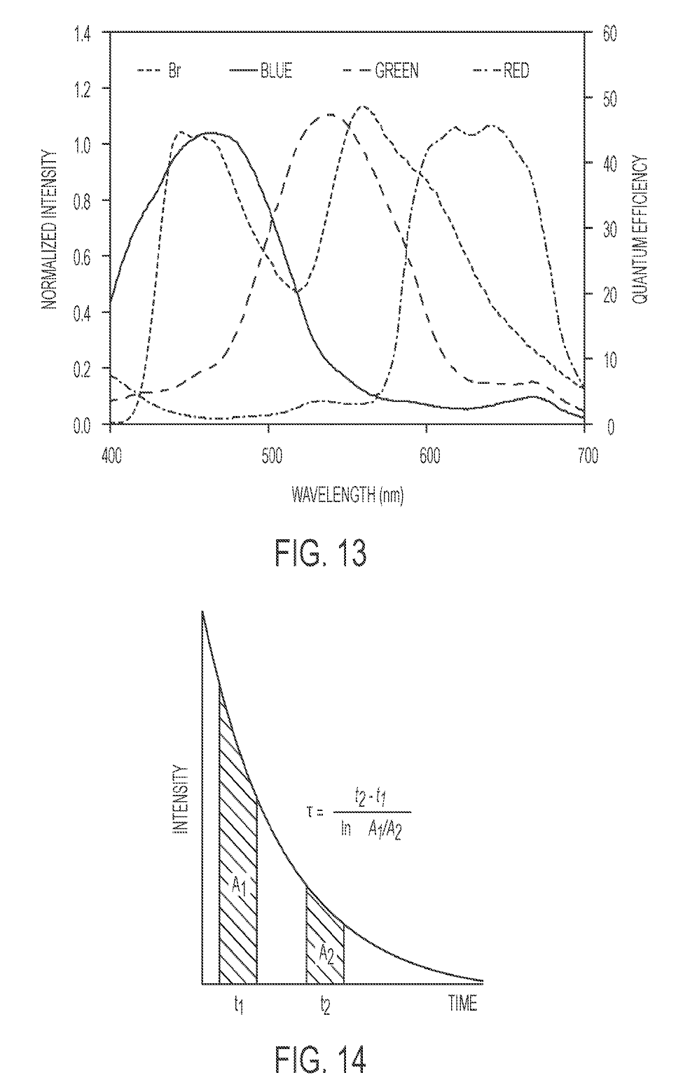

For Br-NP, the phosphorescence intensity was strong enough for ratiometric imaging. The red/green/blue (RGB) color channels of the camera were used to independently monitor changes in F and P for referenced (F/P) oxygen sensing. RGB camera calibration of Br-NP revealed that using the blue channel for the reference (F) and the red channel as the sensor (P) generated the best calibration curve for this material. The green channel was excluded from the measurements to provide the most spectrally isolated features of the material. Regions between the excitation pulse and decay were used to quantify O₂ via RGB, while regions of decay monitored the O₂ via phosphorescence lifetime. FIG. 13 shows the overlay of the total emission of Br-P under N₂ (Br; black dashed line) with the Point Grey GS3 camera Red, Green, and Blue channel quantum efficiencies. Oxygen levels acquired by the two methods were in good agreement with each other. Ratiometric referenced intensity calibration (i.e. comparing intensities at λ_{F} and λ_{RTP} maxima; I_{F}/I_{P}) revealed a linear correlation from 0-1% O₂ with reliable detection to 3%. As shown in FIG. 4, although it is possible to sense oxygen to 21%, changes in the F/P ratio are minimal from 3-21%, making it difficult to distinguish the O₂ level in these ranges. As shown in FIG. 5, when a lifetime calibration is performed on Br-NP, the Stern-Volmer quenching constant (τ_{P} vs O₂) had a linear correlation from 0-21% O₂. At higher oxygen concentrations, the lifetime method provided higher resolution images than the RGB images. Thus, both ratiometry and lifetime methods are suitable for Br-NPs within appropriate [O₂] ranges.

I-NP showed oxygen-sensing capability unprecedented for boron β-dikeonate materials. As shown in FIG. 6B, ratiometry works well for this material, as the O₂ concentration linearly correlated with both the referenced intensity (RI) which measures the F/P from the fluorescence and phosphorescence maxima peaks from the total emission spectra, as well as B/R, which measures the blue channel to red channel intensity. As shown in FIG. 6C, the I-NP exhibits a 0-100% O₂ sensitivity range. the F/P ratio is linearly correlated (R²=0.993). Furthermore, because the RTP is much stronger than F and is never fully quenched even in pure O₂, as shown in FIG. 6A, interference from fluorescence is negligible. Ratiometry works well for this material, while the unquenched lifetime (1.7 ms) is on the edge of the detection limits of the current camera instrumentation (2.0 ms). The reduction in oxygen sensitivity for I-NP dramatically increases potential uses in normoxic tissue (e.g. veins vs arteries: 10 vs 15% O₂).

Table 4 shows the oxygen sensing properties of the nanoparticles.

TABLE 4
Oxygen Sensing Characteristics of Nanoparticles

| | | | |
|---|---|---|---|
| | | Lower | Upper |
| | K_{SV}^{a} | LOD^{b} | LOD^{c} |
| Sample | (O₂%)⁻¹ | (%) | (%) |

| | | | | |
|---|---|---|---|---|
| BF₂nbmPLA | H-NP | 50.87^{d} | 0.01^{d} | 0.75^{d} |
| BF₂nbm(Br)PLA | Br-NP | 2.058^{d} | 0.05^{d} | 21.0^{d} |
| | | 1.875^{e} | 0.05^{e} | 21.0^{e} |
| BF₂nbm(I)PLA | I-NP | 0.024^{e} | 0.50^{e} | 100^{e} |

^{a}Single site Stern-Volmer quenching constant per percent O₂. (F/P₀ ÷ F/P)/% O₂ = P/P₀ per % O₂, where P = phosphorescence intensity and K_{SV} = P/P₀.
^{b}Estimated lower limit of detection defined as τ/τ₀ = 0.99 (i.e. when 1% of phosphorescence is quenched). ¹⁵
^{c}Estimated upper limit of detection defined as τ/τ₀ = 0.01 (i.e. when 99% of phosphorescence is quenched).
^{d}Values based on lifetime calibration data.
^{e}Values based on ratiometric calibration data.

### Example 3

To demonstrate the utility of nanoparticle/camera imaging for biological oxygen sensing, nanoparticles were applied to a murine full thickness skin wound.

Murine Full Thickness Skin Wound Model. All procedures were performed in accordance with the University of Virginia Institutional Animal Care and Use Committee. Female 12-16 week old C57BL/6 mice were used for the studies. A previously published non-splinted full thickness skin wound model was adapted and used for in vivo imaging trials.⁹⁶ Briefly, mice were anesthetized with ketamine/xylazine/atropine (60/4/0.2 mg/kg) and the dorsum of the mice were depilated and sterilized. Mice were laid on their sides and dorsal skin was tented and pinned away from the body of the mouse to create a folded layer of skin. Three, 3 mm equidistant biopsy punches were created through the two layers of skin so as to create six, ˜3 mm full thickness skin wounds. An analgesic (buprenorphine, 0.1 mg/kg) was administered following surgery and the wounds were covered with a Tegaderm dressing.

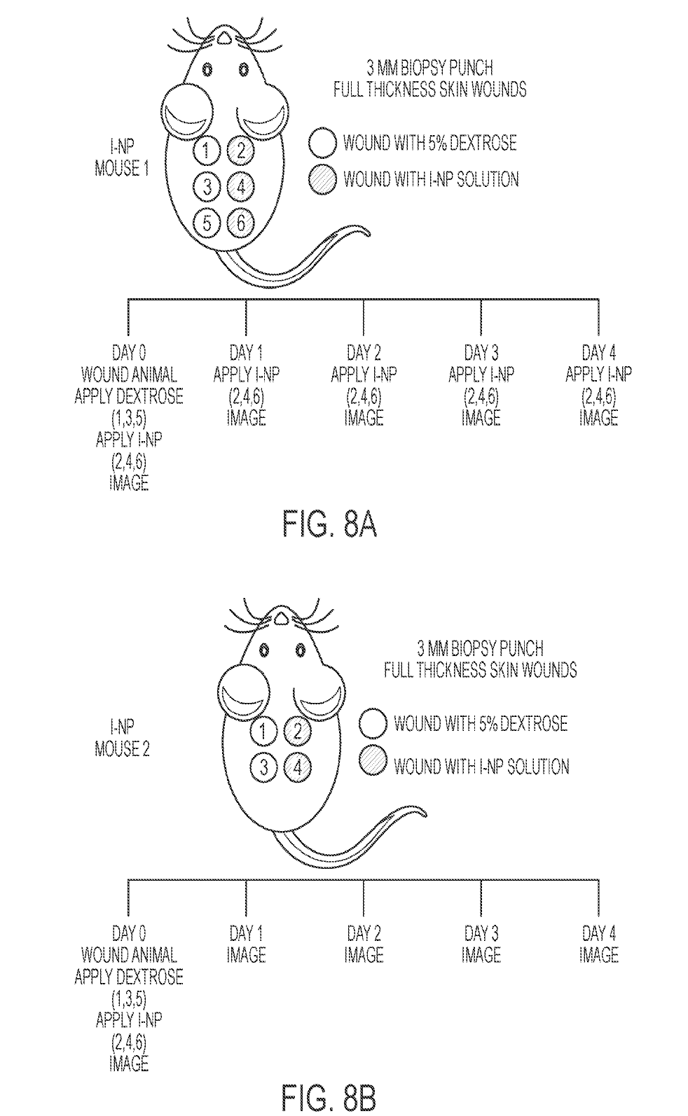

Imaging Procedure. The camera (Point Grey, Grasshopper 3) was mounted to a Nikon Eclipse 80i equipped with an X-Cite 120 fluorescence light source filtered with a bandpass excitation filter (360/20 nm) and a longpass barrier filter (>425 nm, Edmund Optics). Mice were anesthetized with an inhalable 2% isoflurane/oxygen mixture and Tegaderm bandages were removed. Images of each wound under white light were taken using 20× magnification power to quantify wound area. Prior to imaging, wounds 1, 3, and 5 were superfused with 5% dextrose solution (10 μL), while wounds 2, 4, and 6 (right side) were superfused with NPs (10 μL, ˜1 mg/ml solution). For Br-NP and I-NP wounds 2, 4, and 6 were dosed before each imaging session, while H-NP were only dosed on wounds 2, 4, and 6 prior to the first imaging session (day 0). FIG. 8A shows an experimental scheme with daily nanoparticle application. FIG. 8B shows an experimental scheme with single nanoparticle application. M-JPEGs were acquired under UV illumination for each wound of Br-NP and I-NP trial consisting of: 1) 5 frames (acquired at 1 frame/3 seconds) of the wound prior to application of 5% dextrose or NPs, 2) 60 frames (acquired at 1 frame/3 seconds) of wounds after application of 5% dextrose or Br-NPs, and 3) 60 frames (acquired at 1 frame/3 seconds) of the wound after placing a sterile coverslip over the wound to exclude ambient air. For the H-NP trial to test fluorescence retention, single images were acquired for the wounds under white light excitation and fluorescent excitation.

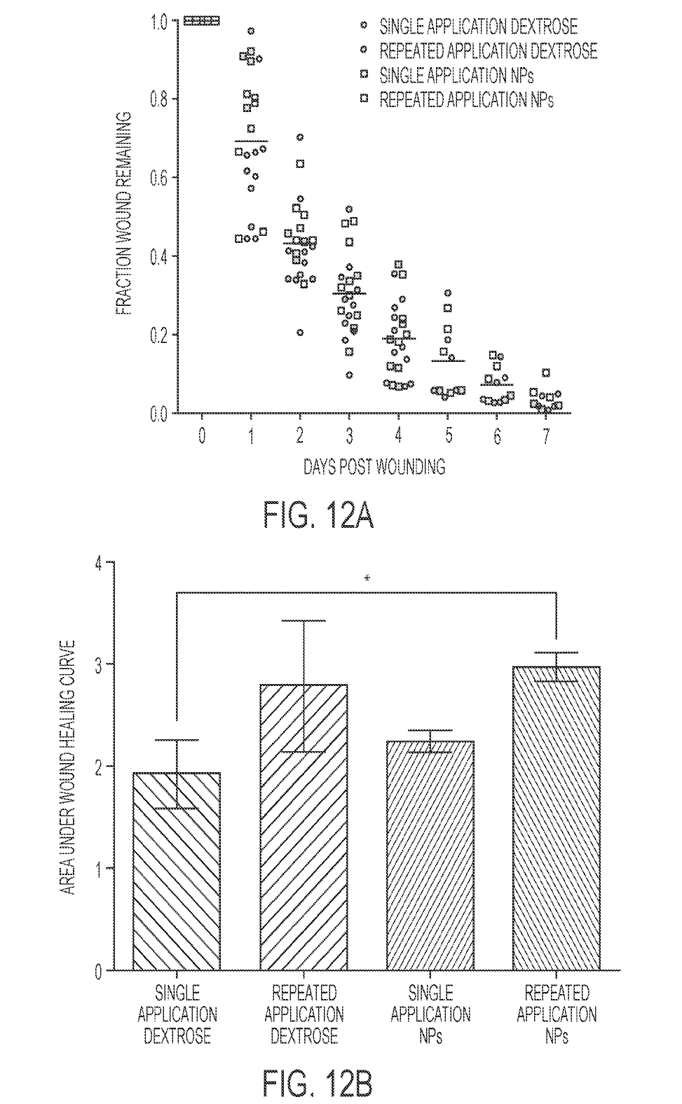

The area under the curve for each treatment was quantified to determine the effect of NPs on wound healing using one-way ANOVA and P<0.05 for single application dextrose and repeated application NPs.⁹ As shown in FIG. 12B, application of the I-NPs did not significantly slow or delay the wound healing process.

Wound Area Quantification. Brightfield, 200× images of wounds were acquired at each time point and were imaged as described above. ImageJ⁹⁷ was used to quantify the wound bed area at each day by manually tracing the wound bed and calculating the area. To keep measurements consistent, the periphery of the wound was traced at the outermost edge of the wound barrier in each image.

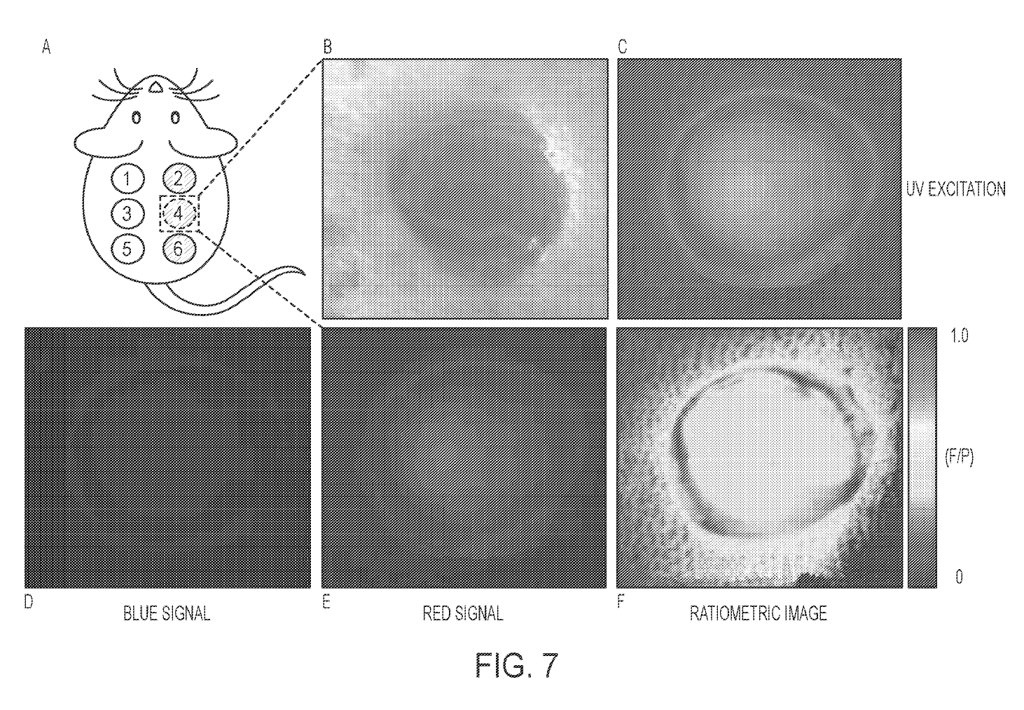

Wound Image Processing. The UV-illuminated wound images (acquired as described above) were analyzed using custom written MATLAB programs. Specific points within the wound bed were selected and the intensities of the red and blue color channels at those points were analyzed over the course of the image series. The background color intensities of the images at those points, at times prior to the addition of nanoparticles, were subtracted from the image series for all points within the image. As a result, any subsequent non-zero values for the red and blue channels were the result of nanoparticle fluorescence and phosphorescence only. The ratio of blue light intensity over red light intensity was computed for each pixel to represent the ratio of blue fluorescence (constant in the presence of NPs) to red phosphorescence (quenched in the presence of oxygen). The upper and lower bounds for this ratio were set according to the different nanoparticle sensitivity ranges. The ratiometric images were then displayed using a 256-value color map scaled to the ratio bounds for spatiotemporally resolving fluorescence-to-phosphorescence ratios (F/P). Panels B through F in FIG. 7 show various images of I-NPs applied to a wound. Upon I-NP application, the wound bed displayed bright yellow emission (strong P+weak F) even without covering the wound. Panel F in FIG. 7 shows the periphery of the wound bed to be highly deprived of oxygen.

Wound Healing. The camera imaging system with I-NP was also used to correlate wound oxygenation with recovery.

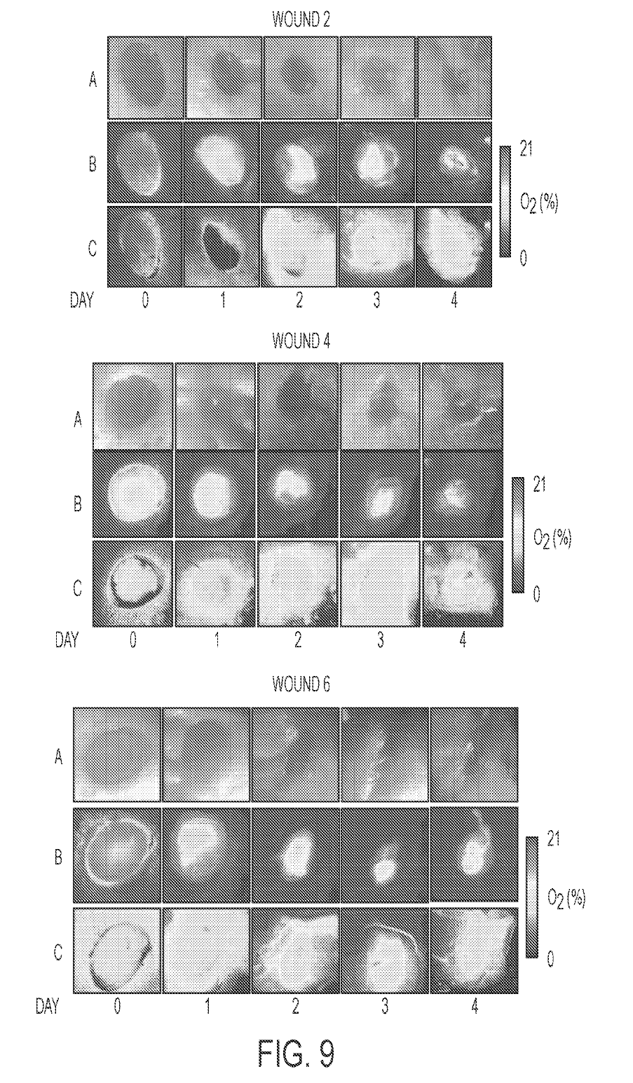

As shown in FIG. 9, I-NP gave consistent measurements of the oxygen levels day to day for covered and uncovered measurements. I-NPs were applied fresh daily. Residual fluorescence from the previous day was removed from the analysis with a background subtraction before fresh NPs were applied. On day 0, the periphery of the I-NP treated wound bed appeared to be highly deprived of oxygen. Individual wound areas decreased over time and oxygenation levels within the wound bed increased up to Day 4. Wound contracture and re-epithelialization began to occur at Day 4, which is consistent with the well-documented wound-healing cascade.

### Example 4

### Comparative Example —Wound Imaging with BF₂dbm(I)PLA

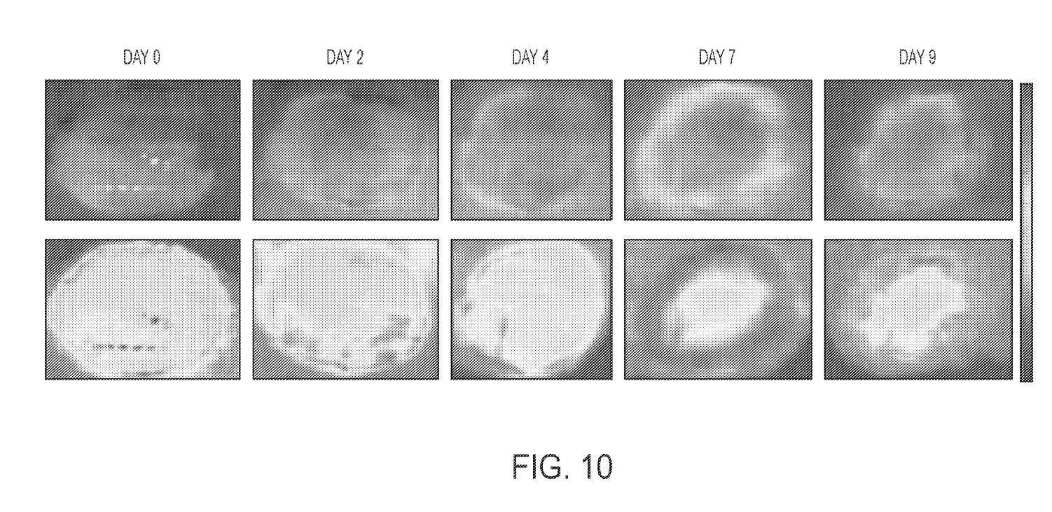

Early generation nanoparticles of iodo-dibenzyl analogue, BF₂dbm(I)PLA,²⁷ were applied to murine skin wounds and monitored for 9 days with a digital single-lens reflex (DSLR) camera equipped with a light-emitting diode camera attachment (LED-ring). The bright fluorescence from a single application on day 1 was still observable in the wound bed for 9 days. However, RTP within the wound bed was weak, and, as shown in FIG. 10, F/P ratios generated from the camera were unclear and difficult to interpret.

### Example 5

### Comparative Example

### Wound Imaging with Br-NP

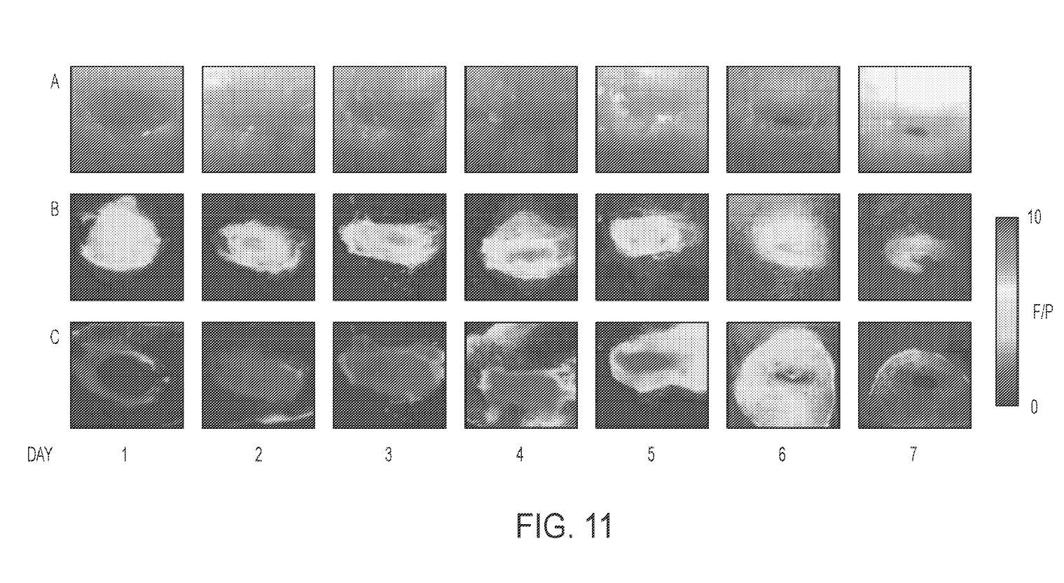

When Br-NPs were applied to the wound, as seen in FIG. 11, ratio changes were barely above the baseline fluorescence. When attempting lifetime mode imaging with Br-NP, no delayed emission was observed, or it was too short-lived for detection (<2 ms). When isolated from air (covered) however, RTP did activate. However, the response of Br-NPs was less dynamic than I-NP, indicating that Br-NPs are not as well suited for wound imaging as I-NP.

### Example 6

Imaging brain tissue with I-NP All procedures were performed in accordance with the University of Virginia Institutional Animal Care and Use Committee. An eight-week-old male C57BL/6 mouse was used for the study. Throughout the experiment, the mouse was maintained under anesthesia with 1.0-1.5% vaporized isoflurane, and the body temperature was kept at 37° C. using a temperature-controlled heating pad (Cole-Parmer, EW-89802-52; Omega, SRFG-303/10). The skin and skull were surgically removed to expose the cortex. 10 μL of 1 mg/mL BNPs suspended in deionized water was topically applied to the exposed cortex, and images/videos were acquired using a CCD camera mounted to a Nikon Eclipse 80i upright microscope under UV excitation. The cortex was left uncovered during imaging. The middle cerebral artery occlusion (MCAO) stroke model was performed, as previously described.⁹⁸ After initiation of the MCAO, 10 μL of 1 mg/mL BNPs was applied topically followed by acquisition of images/videos to quantify oxygenation levels within the tissue while the cortex remained uncovered. A custom-written MATLAB program was used to extract oxygenation data from the acquired images/videos.

Nanoparticles were delivered to the surface of the brain via a murine cranial window that was made through the skull, and ratiometric imaging using ultraviolet (UV) excitation revealed blood vessels in the brain and provided a visual read-out of the amount of oxygen in the brain tissue. Bottom row: I-NP were re-applied to the brain 5 minutes after a stroke was surgically initiated, and the ratiometric imaging of the oxygen-sensing nanoparticles revealed a drastic reduction in oxygen levels in the brain tissue, as evidenced by the blue color in the ratiometric image (bottom right panel).

Having thus described the preferred embodiments of the present invention, those of skill in the art will readily appreciate that the teachings found herein may be applied to yet other embodiments within the scope of the attached claims.

All references cited herein are hereby incorporated by reference and in their entirety.

### REFERENCES

(1) Roussakis, E.; Li, Z.; Nichols, A. J.; Evans, C. L. Oxygen-Sensing Methods in Biomedicine from the Macroscale to the Microscale. Angew. Chem. Int. Ed. 2015, 54, 8340-8362.
(2) Schreml, S.; Szeimies, R. M.; Prantl, L.; Karrer, S.; Landthaler, M.; Babilas, P. Oxygen in Acute and Chronic Wound Healing. Br. J. Dermatol. 2010, /63,257-268.
(3) Peplow, M. A Smarter Bandage. Sci. Am. 2015, 312, 47-49.
(4) Quaranta, M.; Borisov, S. M.; Klimant, I. Indicators for Optical Oxygen Sensors. Bioanal. Rev. 2012, 4, 115-157.
(5) Xu, W.; Lu, S.; Chen, Y.; Zhao, T.; Jiang, Y.; Wang, Y.; Chen, X. Simultaneous Color Sensing of O2 and pH Using a Smartphone. Sensor Actuat. B-Chem. 2015, 220, 326-330.
(6) Meier, R. J.; Fischer, L. H.; Wolfbeis, O. S.; Schäferling, M. Referenced Luminescent Sensing and Imaging with Digital Color Cameras: A Comparative Study. Sensor Actuat. B-Chem. 2013, 177,500-506.
(7) Wang, X.; Wolfbeis, O. S. Optical Methods for Sensing and Imaging Oxygen: Materials, Spectroscopies and Applications. Chem. Soc. Rev. 2014, 43, 3666-3761.
(8) Meier, R. J.; Schreml, S.; Wang, X. D.; Landthaler, M.; Babilas, P.; Wolfbeis, O. S. Simultaneous Photographing of Oxygen and pH in Vivo Using Sensor Films. Angew. Chem. Int. Ed. 2011, 50, 10893-10896.
(9) Schreml, S.; Meier, R. J.; Kirschbaum, M.; Kong, S. C.; Gehmert, S.; Felthaus, O.; Küchler, S.; Sharpe, J. R.; Wöltje, K.; Weiβ, K. T.; Albert, M.; Seidl, U.; Schröder, J.; Morsczeck, C.; Prantl, L.; Duschl, C.; Pedersen, S. F.; Gosau, M.; Berneburg, M.; Wolfbeis, O. S.; Landthaler, M.; Babilas, P. Luminescent Dual Sensors Reveal Extracellular pH-Gradients and Hypoxia on Chronic Wounds That Disrupt Epidermal Repair. Theranostics 2014, 4, 721-735.
(10) Roussakis, E.; Li, Z.; Nowell, N. H.; Nichols, A. J.; Evans, C. L. Bright, “Clickable” Porphyrins for the Visualization of Oxygenation under Ambient Light. Angew. Chem. Int. Ed. 2015, 54, 14728-14731.
(11) Nichols, A. J.; Roussakis, E.; Klein, O. J.; Evans, C. L. Click-Assembled, Oxygen-Sensing Nanoconjugates for Depth-Resolved, near-Infrared Imaging in a 3D Cancer Model. Angew. Chem. Int. Ed. 2014, 53,3671-3674.
(12) Li, Z.; Roussakis, E.; Koolen, P. G. L.; Ibrahim, A. M. S.; Kim, K.; Rose, L. F.; Wu, J.; Nichols, A. J.; Baek, Y.; Birngruber, R.; Apiou-Sbirlea, G.; Matyal, R.; Huang, T.; Chan, R.; Lin, S. J.; Evans, C. L. Non-Invasive Transdermal Two-Dimensional Mapping of Cutaneous Oxygenation with a Rapid-Drying Liquid Bandage. Biomed. Opt. Express 2014, 5, 3748-3764.
(13) Yin, X.; Guo, F.; Lalancette, R. A.; Jäkle, F. Luminescent Main-Chain Organoborane Polymers: Highly Robust, Electron-Deficient Poly(oligothiophene Borane)s via Stille Coupling Polymerization. Macromolecules 2016, 49, 537-546.
(14) Karpenko, I. A.; Niko, Y.; Yakubovskyi, V. P.; Gerasov, A. O.; Bonnet, D.; Kovtun, Y. P.; Klymchenko, A. S. Push-pull Dioxaborine as Fluorescent Molecular Rotor: Far-Red Fluorogenic Probe for Ligand-receptor Interactions. J. Mater. Chem. C 2016, 4, 3002-3009.
(15) Jäkle, F. Advances in the Synthesis of Organoborane Polymers for Optical, Electronic, and Sensory Applications. Chem. Rev. 2010, 110, 3985-4022.
(16) Xiao, H.; Li, P.; Zhang, W.; Tang, B. An Ultrasensitive near-Infrared Ratiometric Fluorescent Probe for Imaging Mitochondrial Polarity in Live Cells and in Vivo. Chem. Sci. 2016, 7, 1588-1593.
(17) Li, D.; Zhang, H.; Wang, Y. Four-Coordinate Organoboron Compounds for Organic Light-Emitting Diodes (OLEDs). Chem. Soc. Rev. 2013, 42, 8416-8433.
(18) Butler, T.; Morris, W. A.; Samonina-Kosicka, J.; Fraser, C. L. Mechanochromic Luminescence and Aggregation Induced Emission of Dinaphthoylmethane β-Diketones and Their Boronated Counterparts. ACS Appl. Mater. Interfaces 2016, 8, 1242-1251.
(19) Morris, W. A.; Liu, T.; Fraser, C. L. Mechanochromic Luminescence of Halide-Substituted Difluoroboron β-Diketonate Dyes. J. Mater. Chem. C 2015, 3, 352-363.
(20) Zhang, G.; Lu, J.; Sabat, M.; Fraser, C. L. Polymorphism and Reversible Mechanochromic Luminescence for Solid-State Difluoroboron Avobenzone. J. Am. Chem. Soc. 2010, 132, 2160-2162.
(21) Wang, L.; Wang, K.; Zou, B.; Ye, K.; Zhang, H.; Wang, Y. Luminescent Chromism of Boron Diketonate Crystals: Distinct Responses to Different Stresses. Adv. Mater. 2015, 27, 2918-2922.
(22) Wong, C.-L.; Poon, C.-T.; Yam, V. W.-W. Photochromic Dithienylethene-Containing Boron(III) Ketoiminates: Modulation of Photo-Responsive Behavior through Variation of Intramolecular Motion. Chem. Eur. J. 2016. 22, 12931-12940.
(23) Mellerup, S. K.; Yuan, K.; Nguyen, C.; Lu, Z.-H.; Wang, S. Donor-Appended N, C-Chelate Organoboron Compounds: Influence of Donor Strength on Photochromic Behaviour. Chem. Eur. J. 2016, 22, 12464-12472.
(24) Poon, C.-T.; Lam, W. H.; Wong, H.-L.; Yam, V. W.-W. A Versatile Photochromic Dithienylethene-Containing β-Diketonate Ligand: Near-Infrared Photochromic Behavior and Photoswitchable Luminescence Properties upon Incorporation of a boron(III) Center. J. Am. Chem. Soc. 2010, 132, 13992-13993.
(25) Cheng, F.; Bonder, E. M.; Jäkle, F. Electron-Deficient Triarylborane Block Copolymers: Synthesis by Controlled Free Radical Polymerization and Application in the Detection of Fluoride Ions. J. Am. Chem. Soc. 2013, 135, 17286-17289.
(26) Xin, L.; Chen, Y.-Z.; Niu, L.-Y.; Wu, L.-Z.; Tung, C.-H.; Tong, Q.-X.; Yang, Q.-Z. A Selective Turn-on Fluorescent Probe for Cd2+ Based on a Boron Difluoride β-Dibenzoyl Dye and Its Application in Living Cells. Org. Biomol. Chem. 2013, 11, 3014-3019.
(27) Zhang, G.; Palmer, G. M.; Dewhirst, M. W.; Fraser, C. L. A Dual-Emissive-Materials Design Concept Enables Tumour Hypoxia Imaging. Nat. Mater. 2009, 8, 747-751.
(28) Lehner, P.; Staudinger, C.; Borisov, S. M.; Klimant, I. Ultra-Sensitive Optical Oxygen Sensors for Characterization of Nearly Anoxic Systems. Nat. Commun. 2014, 5,4460.
(29) Lehner, P.; Staudinger, C.; Borisov, S. M.; Regensburger, J.; Klimant, I. Intrinsic Artefacts in Optical Oxygen Sensors—How Reliable Are Our Measurements? Chem. Eur. J. 2015, 21, 3978-3986.
(30) Zhang, G.; Chen, J.; Payne, S. J.; Kooi, S. E.; Demas, J. N.; Fraser, C. L. Multi-Emissive Difluoroboron Dibenzoylmethane Polylactide Exhibiting Intense Fluorescence and Oxygen-Sensitive Room-Temperature Phosphorescence. J. Am. Chem. Soc. 2007, 129, 8942-8943.
(31) Contreras, J.; Xie, J.; Chen, Y. J.; Pei, H.; Zhang, G.; Fraser, C. L.; Hamm-Alvarez, S. F. Intracellular Uptake and Trafficking of Difluoroboron Dibenzoylmethane-Polylactide Nanoparticles in HeLa Cells. ACS Nano 2010, 4, 2735-2747.
(32) Pfister, A.; Zhang, G.; Zareno, J.; Horwitz, A. F.; Fraser, C. L. Boron Polylactide Nanoparticles Exhibiting Fluorescence and Phosphorescence in Aqueous Medium. ACS Nano 2008, 2, 1252-1258.
(33) Bowers, D. T.; Tanes, M. L.; Das, A.; Lin, Y.; Keane, N. A.; Neal, R. A.; Ogle, M. E.; Brayman, K. L.; Fraser, C. L.; Botchwey, E. A. Spatiotemporal Oxygen Sensing Using Dual Emissive Boron Dye-Polylactide Nanofibers. ACS Nano 2014, 8, 12080-12091.
(34) Samonina-Kosicka, J.; Weitzel, D. H.; Hofmann, C. L.; Hendargo, H.; Hanna, G.; Dewhirst, M. W.; Palmer, G. M.; Fraser, C. L. Luminescent Difluoroboron β-Diketonate PEG-PLA Oxygen Nanosensors for Tumor Imaging. Macromol. Rapid Commun. 2015, 36,694-699.
(35) Kersey, F. R.; Zhang, G.; Palmer, G. M.; Dewhirst, M. W.; Fraser, C. L. Stereocomplexed Poly(lactic Acid)-Poly(ethylene Glycol) Nanoparticles with Dual-Emissive Boron Dyes for Tumor Accumulation. ACS Nano 2010, 4, 4989-4996.
(36) Samonina-Kosicka, J.; DeRosa, C. A; Morris, W. A; Fan, Z.; Fraser, C. L. Dual-Emissive Difluoroboron Naphthyl-Phenyl β-Diketonate Polylactide Materials: Effets of Heavy Atom Placement and Polymer Molecular Weight. Macromolecules 2014, 47, 3736-3746.
(37) DeRosa, C. A.; Samonina-Kosicka, J.; Fan, Z.; Hendargo, H. C.; Weitzel, D. H.; Palmer, G. M.; Fraser, C. L. Oxygen Sensing Difluoroboron Dinaphthoylmethane Polylactide. Macromolecules 2015, 48, 2967-2977.
(38) DeRosa, C. A.; Kerr, C.; Fan, Z.; Kolpaczynska, M.; Mathew, A. S.; Evans, R. E.; Zhang, G.; Fraser, C. L. Tailoring Oxygen Sensitivity with Halide Substitution in Difluoroboron Dibenzoylmethane Polylactide Materials. ACS Appl. Mater. Interfaces 2015, 7, 23633-23643.
(39) Kolpaczynska, M.; DeRosa, C. A.; Morris, W. A.; Fraser, C. L. Thienyl Difluoroboron β-Diketonates in Solution and Polylactide Media. Aust. J. Chem. 2016, 69, 537-545.
(40) Payne, S. J.; Zhang, G.; Demas, J. N.; Fraser, C. L.; Degraff, B. A. Laser Phosphoroscope and Applications to Room-(1) Roussakis, E.; Li, Z.; Nichols, A. J.; Evans, C. L. Oxygen-Sensing Methods in Biomedicine from the Macroscale to the Microscale. Angew. Chem. Int. Ed. 2015, 54, 8340-8362.
(41) Zhang, G.; Evans, R. E.; Campbell, K. a.; Fraser, C. L. Role of Boron in the Polymer Chemistry and Photophysical Properties of Difluoroboron-Dibenzoylmethane Polylactide. Macromolecules 2009, 42, 8627-8633.
(42) Zhang, G.; Kooi, S. E.; Demas, J. N.; Fraser, C. L. Emission Color Tuning with Polymer Molecular Weight for Difluoroboron Dibenzoylmethane-Polylactide. Adv. Mater. 2008, 20 (11), 2099-2104.
(43) Lower, S. K.; EI-Sayed, M. A. The Triplet State and Molecular Electronic Processes in Organic Molecules. Chem. Rev. 1966, 66, 199-241.
(44) Lakowicz, J. R. Principles of Fluorescence Spectroscopy; 2006.
(45) Moβhammer, M.; Strobl, M.; Kühl, M.; Klimant, I.; Borisov, S. M.; Koren, K. Design and Application of an Optical Sensor for Simultaneous Imaging of pH and Dissolved O₂ with Low Cross-Talk. ACS Sensors 2016, 1, 681-687.
(46) Baggaley, E.; Botchway, S. W.; Haycock, J. W.; Morris, H.; Sazanovich, I. V; Williams, J. A. G.; Weinstein, J. A. Long-Lived Metal Complexes Open up Microsecond Lifetime Imaging Microscopy under Multiphoton Excitation: From FLIM to PLIM and beyond. Chem. Sci. 2014, 5, 879-886.
(47) Sakadžić, S.; Roussakis, E.; Yaseen, M. A.; Mandeville, E. T.; Srinivasan, V. J.; Arai, K.; Ruvinskaya, S.; Devor, A.; Lo, E. H.; Vinogradov, S. A.; Boas, D. A. Two-Photon High-Resolution Measurement of Partial Pressure of Oxygen in Cerebral Vasculature and Tissue. Nat. Methods 2010, 7, 755-759.
(48) Lebedev, A. Y.; Cheprakov, A. V.; Sakadzic, S.; Boas, D. a.; Wilson, D. F.; Vinogradov, S. a. Dendritic Phosphorescent Probes for Oxygen Imaging in Biological Systems. ACS Appl. Mater. Interfaces 2009, 1, 1292-1304.
(49) Mathew, A. S.; DeRosa, C. A.; Demas, J. N.; Fraser, C. L. Difluoroboron β-Diketonate Materials with Long-Lived Phosphorescence Enable Lifetime Based Oxygen Imaging with a Portable Cost Effective Camera. Anal. Methods 2016, 8, 3109-3114.
(50) Bishop, A. Role of Oxygen in Wound Healing. J. Wound Care 2008, 17, 399-402.
(51) Branson, R. D.; Johannigman, J. A. Pre-Hospital Oxygen Therapy. Respir. Care 2013, 58, 86-97.
(52) Giaccia, A. J.; Simon, M. C.; Johnson, R. The Biology of Hypoxia: The Role of Oxygen Sensing in Development, Normal Function, and Disease. Genes Dev. 2004, 18, 2183-2194.
(53) Zhang, G.; Fiore, G. L.; Clair, T. L. S.; Fraser, C. L. Difluoroboron Dibenzoylmethane Pcl-Pla Block Copolymers: Matrix Effects on Room Temperature Phosphorescence. Macromolecules 2009, 42, 3162-3169.
(54) Tandara, A. A.; Mustoe, T. A. Oxygen in Wound Healing—More than a Nutrient. World J. Surg. 2004, 28, 294-300.
(55) Wang, X.; Meier, R. J.; Schmittlein, C.; Schreml, S.; Schäferling, M.; Wolfbeis, O. S. A Water-Sprayable, Thermogelating and Biocompatible Polymer Host for Use in Fluorescent Chemical Sensing and Imaging of Oxygen, pH Values and Temperature. Sensor Actuat. B-Chem. 2015, 221, 37-44.
(56) Hofmann, J.; Meier, R. J.; Mahnke, A.; Schatz, V.; Brackmann, F.; Trollmann, R.; Bogdan, C.; Liebsch, G.; Wang, X.; Wolfbeis, O. S.; Jantsch, J. Ratiometric Luminescence 2D in Vivo Imaging and Monitoring of Mouse Skin Oxygenation. Methods Appl. Fluoresc. 2013, 1, 45002.
(57) Babilas, P.; Lamby, P.; Prantl, L.; Schreml, S.; Jung, E. M.; Liebsch, G.; Wolfbeis, O. S.; Landthaler, M.; Szeimies, R.-M.; Abels, C. Transcutaneous pO₂ Imaging during Tourniquet-Induced Forearm Ischemia Using Planar Optical Oxygen Sensors. Ski. Res. Technol. 2008, 14, 304-311.
(58) Schreml, S.; Meier, R. J.; Wolfbeis, O. S.; Maisch, T.; Szeimies, R.-M. M.; Landthaler, M.; Regensburger, J.; Santarelli, F.; Klimant, I.; Babilas, P. 2D Luminescence Imaging of Physiological Wound Oxygenation. Exp. Dermatol. 2011, 20, 550-554.
(59) Demas, J. N.; Crosby, G. A. The Measurement of Photoluminescence Quantum Yields. A Review. J. Phys. Chem. 1971, 75,991-1024.
(60) Kochmann, S.; Baleizão, C.; Berberan-Santos, M. N.; Wolfbeis, O. S. Sensing and Imaging of Oxygen with Parts per Billion Limits of Detection and Based on the Quenching of the Delayed Fluorescence of 13C 70 Fullerene in Polymer Hosts. Anal. Chem. 2013, 85, 1300-1304.
(61) A. J. Giaccia, M. C. Simon and R. Johnson, Genes Dev., 2004, 18, 2183-94.
(62) X. Wang and O. S. Wolfbeis, Chem. Soc. Rev., 2014, 43, 3666-761.
(63) R. Y. Kannan, H. J. Salacinski, K. Sales, P. Butler and A. M. Seifalian, Biomaterials, 2005, 26, 1857-1875.
(64) E. A. Phelps and A. J. Garcia, Curr. Opin. Biotechnol., 2010, 21, 704-9.
(65) M. Hockel and P. Vaupel, JNCI J. Natl. Cancer Inst., 2001, 93, 266-276.
(66) A. L. Harris, Nat. Rev. Cancer, 2002, 2, 38-47.
(67) A. Bishop, J. Wound Care, 2008, 17, 399-402.
(68) X. F. Wang, S. Kitajima, T. Uchida, D. M. Coleman and S. Minami, Appl. Spectrosc., 1990, 44, 25-30.
(69) X. F. Wang, T. Uchida, D. M. Coleman and S. Minami, Appl. Spectrosc., 1991, 45, 360-366.
(70) X. F. Wang, T. Uchida and S. Minami, Appl. Spectrosc., 1989, 43, 840-845.
(71) R. D. Shonat, D. F. Wilson, C. E. Riva and M. Pawlowski, Appl. Opt., 1992, 31, 3711-8.
(72) G. Liebsch, I. Klimant, B. Frank, G. Hoist and O. S. Wolfbeis, Appl. Spectrosc., 2000, 54, 548-559.
(73) Z. Li, E. Roussakis, P. G. L. Koolen, A. M. S. Ibrahim, K. Kim, L. F. Rose, J. Wu, A. J. Nichols, Y. Baek, R. Birngruber, G. Apiou-Sbirlea, R. Matyal, T. Huang, R. Chan, S. J. Lin and C. L. Evans, Biomed. Opt. Express, 2014, 5, 3748-64.
(74) K. Hanaoka, K. Kikuchi, S. Kobayashi and T. Nagano, J. Am. Chem. Soc., 2007, 129, 13502-9.
(75) Q. Zhao, C. Huang and F. Li, Chem. Soc. Rev., 2011, 40, 2508-24.
(76) Y. Gong, L. Zhao, Q. Peng, D. Fan, W. Z. Yuan, Y. Zhang and B. Z. Tang, Chem. Sci., 2015, 6, 4438-4444.
(77) Z. An, C. Zheng, Y. Tao, R. Chen, H. Shi, T. Chen, Z. Wang, H. Li, R. Deng, X. Liu and W. Huang, Nat. Mater., 2015, 14, 685-90.
(78) O. Bolton, K. Lee, H.-J. Kim, K. Y. Lin and J. Kim, Nat. Chem., 2011, 3, 205-10.
(79) P. Lehner, C. Staudinger, S. M. Borisov, J. Regensburger and I. Klimant, Chemistry, 2015, 21, 3978-86.
(80) P. Lehner, C. Staudinger, S. M. Borisov and I. Klimant, Nat. Commun., 2014, 5, 4460.
(81) G. Zhang, J. Chen, S. J. Payne, S. E. Kooi, J. N. Demas and C. L. Fraser, Macromolecules, 2015, 48, 2967-2977.
(82) D. T. Bowers, M. L. Tanes, A. Das, Y. Lin, N. A. Keane, R. A. Neal, M. E. Ogle, K. L. Brayman, C. L. Fraser and E. A. Botchwey, ACS Nano, 2014, 8, 12080-91.
(83) O. S. Wolfbeis, BioEssays, 2015, 37, 921-928.
(84) R. J. Meier, L. H. Fischer, O. S. Wolfbeis and M. Schäferling, Sensors Actuators B Chem., 2013, 177, 500-506.
(85) A. Ashworth, R. J. Woods, S. Scypinski and L. J. C. Love, Anal. Chem., 1984, 56, 1395-1400.
(86) R. M. Ballew and J. N. Demas, Anal. Chem., 1989, 61, 30-33.
(87) E. R. Carraway, J. N. Demas and B. A. DeGraff, Anal. Chem., 1991, 63, 332-336.
(88) S. J. Payne, G. Zhang, J. N. Demas, C. L. Fraser and B. A. Degraff, Appl. Spectrosc., 2011, 65, 1321-1324.
(89) S. Kochmann, C. Baleizão, M. N. Berberan-Santos and O. S. Wolfbeis, Anal. Chem., 2013, 85, 1300-1304.
(90) T. Palmeira, A. Fedorov and M. N. Berberan-Santos, Methods Appl. Fluoresc., 2014, 2, 035002.
(91) C. A. Kerr and R. de la Rica, Anal. Methods, 2015, 7, 7067-7075.
(92) S. O'Driscoll, B. D. MacCraith and C. S. Burke, Anal. Methods, 2013, 5, 1904.
(93) Irvine, M. W.; Costa, B. M.; Dlaboga, D.; Culley, G. R.; Hulse, R.; Scholefield, C. L.; Atlason, P.; Fang, G.; Eaves, R.; Morley, R.; Mayo-Martin, M. B.; Amici, M.; Bortolotto, Z. A.; Donaldson, L.; Collingridge, G. L.; Molnár, E.; Monaghan, D. T.; Jane, D. E. Piperazine-2,3-Dicarboxylic Acid Derivatives as Dual Antagonists of NMDA and GluK1-Containing Kainate Receptors. J. Med. Chem. 2012, 55 (1), 327-341.
(94) Bender, J. L.; Corbin, P. S.; Fraser, C. L.; Metcalf, D. H.; Richardson, F. S.; Thomas, E. L.; Urbas, A. M. Site-Isolated Luminescent Europium Complexes with Polyester Macroligands: Metal-Centered Heteroarm Stars and Nanoscale Assemblies with Labile Block Junctions. J. Am. Chem. Soc. 2002, 124 (29), 8526-8527.
(95) Williams, D. B. G.; Lawton, M. Drying of Organic Solvents: Quantitative Evaluation of the Efficiency of Several Desiccants. J. Org. Chem. 2010, 75, 8351-8354.
(96) Liu, M.; Saeki, K.; Matsunobu, T.; Okuno, T.; Koga, T.; Sugimoto, Y.; Yokoyama, C.; Nakamizo, S.; Kabashima, K.; Narumiya, S.; Shimizu, T.; Yokomizo, T. 12-Hydroxyheptadecatrienoic Acid Promotes Epidermal Wound Healing by Accelerating Keratinocyte Migration via the BLT2 Receptor. J. Exp. Med. 2014, 211, 1063-1078.
(97) Schneider, C. A.; Rasband, W. S.; Eliceiri, K. W. NIH Image to ImageJ: 25 Years of Image Analysis. Nat. Methods 2012, 9, 671-675.
(98) Vital, S. A., Gavins, F. N. Surgical Approach for Middle Cerebral Artery Occlusion and Reperfusion Induced Stroke in Mice. J. Vis. Exp. (116), e54302, doi:10.3791/54302 (2016).

1. A luminescent dye compound of Formula I: 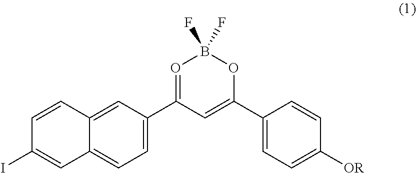
wherein R selected from the group consisting of H, (C₁-C₁₂)alkyl, (C₃-C₁₂)cycloalkyl, (C₁-C₁₀)alkoxy, (C₂-C₁₂)alkenyl, (C₂-C₁₂)alkynyl, (C₁-C₁₂)alkanoyl, (C₁-C₁₂)haloalkyl, (C₁-C₁₂)hydroxyalkyl, (C₁-C₁₂)alkoxycarbonyl, (C₁-C₁₂)alkylthio, (C₂-C₁₂)alkanoyloxy, (C₆-C₂₂)aryl, (C₅-C₁₃)heteroaryl, a polymeric group or combinations thereof.

2. The compound of claim 1, wherein R is a polymeric group, and is selected from the group consisting of polylactide, polyglycolide, poly(ethylene glycol), polycaprolactone, lactide-glycolide copolymer, poly(ethylene glycol)-polylactide, polycaprolactone-polylactide, poly(ethylene glycol)-polycaprolactone poly(ethylene glycol)-polylactide-co-glycolide block copolymers, or a mixture thereof.

3. A composition comprising a compound of claim 1, and a solvent or additional polymer.

4. The composition of claim 3, wherein the compound is dispersed within an additional polymer.

5. The composition of claim 4, wherein the additional polymer is selected from the group consisting of polylactide, polyglycolide, poly(ethylene glycol), polycaprolactone, lactide-glycolide copolymer, poly(ethylene glycol)-polylactide, polycaprolactone-polylactide, poly(ethylene glycol)-polycaprolactone poly(ethylene glycol)-polylactide-co-glycolide block copolymers, or a mixture thereof.

6. The composition of claim 3, wherein the compound is in the form of particles, nanoparticles, films, coatings, fibers or nanofibers, powders, foams, gels, network, assembly, suspension or composite, or bulk material.

7. A method for determining oxygenation levels on a surface comprising the steps of:
(a) contacting the surface with a compound of claim 1 under ambient atmospheric conditions;
(b) exposing the compound on the surface to an excitation source under ambient atmospheric conditions;
(c) detecting the fluorescence and phosphorescence of the compound on the surface under ambient atmospheric conditions; and
(d) determining oxygenation levels on the surface based on the ratio of fluorescence to phosphorescence of the compound.

8. The method of claim 7, wherein the fluorescence and phosphorescence of the compound are detected with a digital camera.

9. The method of claim 8, wherein the digital camera has red/green/blue channels, and wherein the ratio of fluorescence to phosphorescence is measured as the relative intensity of the blue channel to red channel.

10. The method of claim 7 wherein the surface is a mammalian tissue surface.

11. The method of claim 7 wherein the surface is a wound.

12. The method of claim 7, wherein the surface is brain tissue.

13. A method of monitoring wound healing over one or more days, by determining oxygenation levels on the uncovered wound, comprising
(a) contacting the uncovered wound with a compound of claim 1;
(b) exposing the compound on the uncovered wound to an excitation source;
(c) detecting the fluorescence and phosphorescence of the compound on the uncovered wound; and
(d) determining oxygenation levels of the wound based on the ratio of fluorescence to phosphorescence of the compound.

14. The method of claim 13 wherein steps (a)-(d) are performed daily.

15. The method of clam 14 wherein residual fluorescence from the previous day is removed from the measurement with a background subtraction.

16. The method of claim 13, wherein the fluorescence and phosphorescence of the compound are detected with a digital camera.

17. The method of claim 16, wherein the digital camera has red/green/blue channels, and wherein the ratio of fluorescence to phosphorescence is measured as the relative intensity of the blue channel to red channel.
